(19) United States
(12) Patent Application Publication
(10) Pub. No.: US 2024/0319190 A1
(43) Pub. Date: Sep. 26, 2024

(54) CHROMATOGRAPHY DEVELOPING SOLUTION, KIT, CHROMATOGRAPH DEVICE, AND METHOD FOR DETECTING TEST SUBSTANCE

(71) Applicant: KANEKA CORPORATION, Osaka (JP)

(72) Inventor: Ryogo Higashi, Hyogo (JP)

(73) Assignee: KANEKA CORPORATION, Osaka (JP)

(21) Appl. No.: 18/734,211

(22) Filed: Jun. 5, 2024

Related U.S. Application Data
(63) Continuation of application No. PCT/JP2022/044748, filed on Dec. 5, 2022.

(30) Foreign Application Priority Data
Dec. 7, 2021 (JP) 2021-198213

(51) Int. Cl. G01N 33/569; G01N 33/543

(52) U.S. Cl. CPC G01N 33/56983; G01N 33/54346; G01N 33/54387; G01N 2333/165; G01N 2469/10

(57) ABSTRACT

A chromatographic developing solution, a kit including a chromatographic developing solution and a chromatograph device, a chromatograph device, and a method for detecting an analyte contained in a sample are provided. The chromatographic developing solution includes an alkylene oxide-addition cationic surfactant and a nonionic surfactant. The alkylene oxide-addition cationic surfactant includes an ethylene oxide-addition cationic surfactant.

### TECHNICAL FIELD

One or more embodiments of the present disclosure relates to a chromatographic developing solution, a kit, a chromatograph device, and a method for detecting an analyte.

### BACKGROUND

In recent years, many testing techniques have been developed as diagnostic approaches to viral infection, in which techniques the principle of chromatography is applied. Specifically, there is a known testing technique in which a sample possibly containing an analyte, and called a mobile phase passes over the surface of, or through the inside of, a substance called a solid-phase support, and in this process, the specific interaction between the substance of the solid-phase support and the analyte in the mobile phase is utilized to detect the analyte in the sample. Examples of such a testing technique include immunochromatography based on utilizing an immunochromatographic method and nucleic acid chromatography based on utilizing a nucleic acid chromatographic method.

When an antigen as an analyte in a sample is detected in sandwich form by an immunochromatographic method, for example, the following operation is performed.
(1) A trapping substance (immobilizing reagent), for example, an antibody to bind specifically to an antigen as an analyte is, for example, applied at a predetermined position on a solid-phase support to form a trapping substance-retaining section.
(2) In parallel, a detecting reagent such as an antibody to bind specifically to an antigen as an analyte is formed into a composite together with a gold colloid or the like to afford a labeling substance.
(3) A specimen containing an antigen as an analyte is, for example, diluted suitably with a developing solution or the like to prepare a sample, which is developed together with the labeling substance on the solid-phase support.

Through the above-described operation, the composite of the labeling substance and the antigen is developed on a stationary phase, and the antigen binds to the trapping substance in the trapping substance-retaining section, thus being trapped. The operation results in generating a sandwich type of composite formed with three components, namely, the labeling substance, the analyte (antigen), and the trapping substance, in the trapping substance-retaining section, and the analyte is detected.

For example, Patent Literature 1 discloses a medium composition for preparing a suspended-specimen solution, with which composition a false-positive reaction due to a nonspecific reaction can be prevented in an immunoassay such as immunochromatography. As disclosed in Patent Literature 1, the medium composition for preparing a suspended-specimen solution contains an ionic surfactant, is used for an immunoassay, and may further contain a nonionic surfactant.
Patent Literature 1: JP2005-291783A

### SUMMARY

The present inventor has vigorously made studies, aiming to further improve a testing technique based on applying the principle of chromatography, and has consequently discovered that allowing an alkylene oxide-addition cationic surfactant and a nonionic surfactant to act on a labeling substance can achieve high-sensitivity chromatography.

One or more embodiments of the present disclosure provide a chromatographic developing solution that can achieve high-sensitivity chromatography.

One or more embodiments provide: a kit including a chromatographic developing solution and a chromatograph device; a chromatograph device; and a method for detecting an analyte contained in a sample; wherein these can achieve high-sensitivity chromatography.

The present disclosure encompasses the following embodiments.
[1] A chromatographic developing solution including an alkylene oxide-addition cationic surfactant and a nonionic surfactant.
[2] The chromatographic developing solution according to [1], wherein the alkylene oxide-addition cationic surfactant includes an ethylene oxide-addition cationic surfactant.
[3] The chromatographic developing solution according to [1] or [2], wherein the alkylene oxide-addition cationic surfactant includes at least one surfactant selected from a surfactant represented by the following general formula (I) or a surfactant represented by the following general formula (II):

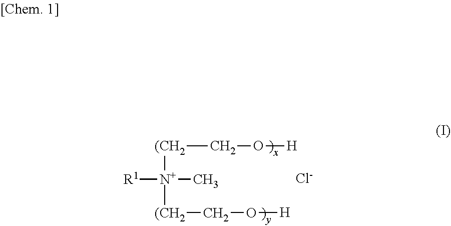

(wherein, in the general formula (I), R¹ is a C₁₋₃₀ saturated or unsaturated hydrocarbon group, x and y are each independently an integer of 1 to 49, and x+y is 2 to 50), and

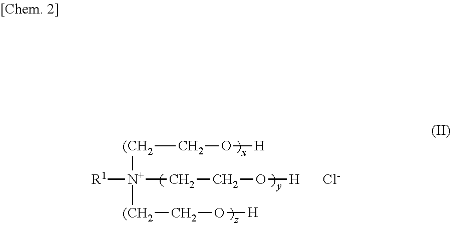

(wherein, in the general formula (II), R² is a C₁₋₃₀ saturated or unsaturated hydrocarbon group, x, y, and z are each independently an integer of 1 to 48, and x+y+z is 3 to 50).
[4] The chromatographic developing solution according to any one of [1] to [3], wherein the alkylene oxide-addition cationic surfactant includes at least one surfactant selected from PEG-5 stearyl ammonium chloride, PEG-2 oleammonium chloride, PEG-2 cocomonium chloride, PEG-15 cocomonium chloride, or PEG-15 steamonium chloride.
[5] The chromatographic developing solution according to any one of [1] to [4], wherein the nonionic surfactant includes at least one surfactant selected from a surfactant represented by the following general formula (III), a surfactant represented by the following general formula (IV), a surfactant represented by the following general formula (V), a surfactant represented by the following general formula (VI), a surfactant represented by the following general formula (VII), or a surfactant represented by the following general formula (VIII):

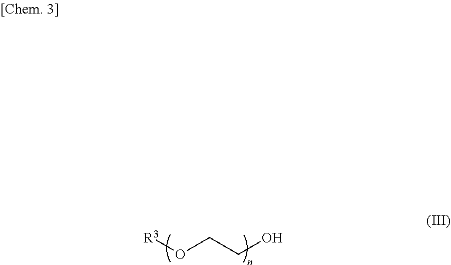

(wherein, in the general formula (III), R³ is a C₁₋₃₀ saturated or unsaturated hydrocarbon group, and n is an integer of 1 to 50),

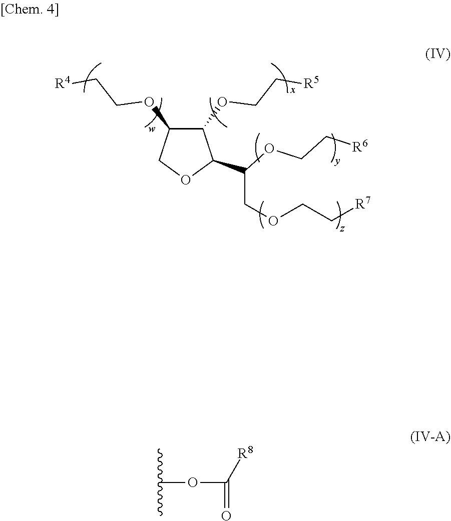

(wherein, in the general formula (IV), R⁴ to R⁷ are each independently a group represented by the above-described general formula (IV-A) or a hydroxy group; at least one of R⁴ to R⁷ is a group represented by the above-described general formula (IV-A); at least one of R⁴ to R⁷ is a hydroxy group;
w, x, y, and z are each independently an integer of 1 to 47, and w+x+y+z is 4 to 50; and
   in the general formula (IV-A), R⁸ is a C₁₋₃₀ saturated or unsaturated hydrocarbon group; and the wavy line represents a binding site to a carbon atom),

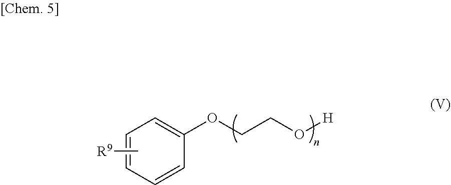

(wherein, in the general formula (V), R⁹ is a C₁₋₃₀ saturated or unsaturated hydrocarbon group, and n is an integer of 1 to 50),

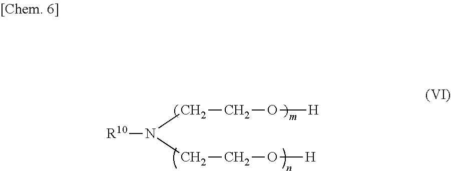

(wherein, in the general formula (VI), R¹⁰ is a C₁₋₃₀ saturated or unsaturated hydrocarbon group, m and n are each independently an integer of 1 to 49, and m+n is 2 to 50).

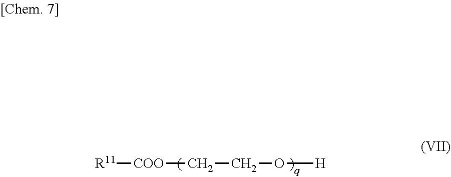

(wherein, in the general formula (VII), R¹¹ is a C₁₋₃₀ saturated or unsaturated hydrocarbon group, and q is an integer of 1 to 300), and

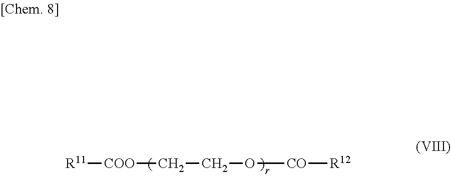

(wherein, in the general formula (VIII), R¹¹ and R¹² are each independently a C₁₋₃₀ saturated or unsaturated hydrocarbon group, and r is an integer of 1 to 300).
[6] The chromatographic developing solution according to any one of [1] to [5], wherein the nonionic surfactant/the alkylene oxide-addition cationic surfactant (as a weight ratio), which is the ratio of the amount of the nonionic surfactant to the amount of the alkylene oxide-addition cationic surfactant, is 0.14 or more and 40 or less.
[7] The chromatographic developing solution according to any one of [1] to [6], including the alkylene oxide-addition cationic surfactant at 0.05 wt % or more and 14 wt % or less.
[8] The chromatographic developing solution according to any one of [1] to [7], including the nonionic surfactant at 0.6 wt % or more and 12 wt % or less.
[9] The chromatographic developing solution according to any one of [1] to [8], wherein the HLB value of the alkylene oxide-addition cationic surfactant is 22 or more and 31 or less.
[10] The chromatographic developing solution according to any one of [1] to [9], which is a specimen diluent.
[11] The chromatographic developing solution according to [10], wherein the specimen is nasal discharge, liquid wiped off the nasal cavity, liquid wiped off the nasopharynx, liquid wiped off the pharynx, or sputum.
[12] The chromatographic developing solution according to any one of [1] to [11], the nonionic surfactant includes at least one surfactant selected from a polyoxyethylenealkyl ether, polyoxyethylenesorbitan fatty acid ester, polyoxyethylenealkylphenyl ether, polyoxyethylenealkyl amine, or polyethylene glycol fatty acid ester.
[13] The chromatographic developing solution according to any one of [1] to [12], wherein the HLB value of the nonionic surfactant is 10 or more and 19 or less.
[14] The chromatographic developing solution according to any one of [1] to [13], further including a nonspecific-adsorption inhibitor.
[15] The chromatographic developing solution according to any one of [1] to [14], further including a dispersibility improver.
[16] The chromatographic developing solution according to any one of [1] to [15], further including an interference inhibitor.
[17] A kit for detecting an analyte, including: the chromatographic developing solution according to any one of [1] to [16]; and a chromatograph device including a sample-receiving section, a labeling substance-retaining section, and a solid-phase support,
wherein the labeling substance-retaining section includes a labeling substance bondable to an analyte, and
wherein the solid-phase support includes a trapping substance-retaining section containing a trapping substance bondable to the analyte.
[18] A kit for detecting an analyte, including: a chromatographic developing solution; and a chromatograph device including a sample-receiving section, a labeling substance-retaining section, and a solid-phase support, wherein
the labeling substance-retaining section includes a labeling substance bondable to an analyte,
the solid-phase support includes a trapping substance-retaining section containing a trapping substance bondable to the analyte, and
an alkylene oxide-addition cationic surfactant and a nonionic surfactant are each independently included in at least one selected from the chromatographic developing solution,
the sample-receiving section, or the labeling substance-retaining section.
[19] The kit according to or [18], wherein the labeling substance includes colored particles.
[20] The kit according to any one of to [19],
wherein the chromatograph device is a chromatograph device including a sample pad as the sample-receiving section, a conjugate pad as the labeling substance-retaining section, and a membrane as the solid-phase support, in this order from upstream in the sample flow direction.
[21] A chromatograph device including a sample-receiving section, a labeling substance-retaining section, and a solid-phase support,
wherein the labeling substance-retaining section includes a labeling substance bondable to an analyte, wherein
the solid-phase support includes a trapping substance-retaining section containing a trapping substance bondable to the analyte, and
an alkylene oxide-addition cationic surfactant and a nonionic surfactant are each independently included in at least one selected from the sample-receiving section or the labeling substance-retaining section.
[22] A method for detecting an analyte contained in a sample, including the following steps (1) and (2):
(1) a step of developing a mobile phase in the presence of an alkylene oxide-addition cationic surfactant and a nonionic surfactant in a solid-phase support of a chromatograph device, the mobile phase including the sample and a labeling substance bondable to an analyte; and
(2) a step of detecting the analyte in the developed mobile phase in a trapping substance-retaining section including a trapping substance bondable to the analyte contained in the solid-phase support.

The contents of the disclosures in Japanese Patent Application No. 2021-198213 which form the basis for priority of the present application are incorporated herein.

According to one or more embodiments of the present disclosure, high-sensitivity chromatography can be achieved by using: a chromatographic developing solution, a kit including a chromatographic developing solution and a chromatograph device, a chromatograph device, and a method for detecting an analyte contained in a sample.

### BRIEF DESCRIPTION OF DRAWINGS

FIG. 1 illustrates schematic views of one example of a chromatograph device; FIG. 1A is a plan view of one example of the chromatograph device; and FIG. 1B is a cross-sectional view of one example of the chromatograph device.

FIG. 2 is a graph illustrating the relationship between the color development intensity of a positive sample and the time elapsed, wherein the sample was produced in each of Examples 16 to 18 and Comparative Examples 20 to 22.

FIG. 3 is a graph illustrating the relationship between the color development intensity of a negative sample and the time elapsed, wherein the sample was produced in each of Examples 16 to 18 and Comparative Examples 20 to 22.

FIG. 4 is a graph illustrating the relationship between the color development intensity of a positive sample and the time elapsed, wherein the sample was produced in each of Examples 49 to 57.

FIG. 5 is a graph illustrating the relationship between the color development intensity of a positive sample and the time elapsed, wherein the sample was produced in each of Example 58 and Comparative Example 25.

FIG. 6 is the photographs each illustrating the external appearance of the immunochromatographic device in which the negative sample or positive sample produced in each of Examples 74 to 81 was developed.

FIG. 7 is the photographs each illustrating the external appearance of the immunochromatographic device in which the negative sample produced in each of Examples 82 to 85 was developed.

### DETAILED DESCRIPTION

One or more embodiments of the present invention will be described in detail below.

A chromatographic developing solution according to one or more embodiments of the present invention includes an alkylene oxide-addition cationic surfactant and a nonionic surfactant. The chromatographic developing solution according to the embodiment is also referred to as a chromatographic developing solution of the embodiment A. The chromatographic developing solution of the embodiment A includes an alkylene oxide-addition cationic surfactant and a nonionic surfactant in the developing solution, and thus, bringing the developing solution into contact with a labeling substance or a composite of an analyte and a labeling substance enables the alkylene oxide-addition cationic surfactant and the nonionic surfactant to act on the labeling substance, and can achieve high-sensitivity chromatography.

One aspect of a kit according to one or more embodiments of the present invention is a kit for detecting an analyte, including: the chromatographic developing solution of the embodiment A; and a chromatograph device including a sample-receiving section, a labeling substance-retaining section, and a solid-phase support, wherein the labeling substance-retaining section includes a labeling substance bondable to an analyte, and wherein the solid-phase support includes a trapping substance-retaining section containing a trapping substance bondable to the analyte. The kit of the embodiment is also referred to as a kit of the embodiment B.

Another aspect of a kit according one or more embodiments of the present invention is, for example, a kit for detecting an analyte, including: a chromatographic developing solution; and a chromatograph device including a sample-receiving section, a labeling substance-retaining section, and a solid-phase support, wherein the labeling substance-retaining section includes a labeling substance bondable to an analyte, wherein the solid-phase support includes a trapping substance-retaining section containing a trapping substance bondable to the analyte, and wherein an alkylene oxide-addition cationic surfactant and a nonionic surfactant are each independently included in at least one selected from the chromatographic developing solution, the sample-receiving section, or the labeling substance-retaining section. The kit of the embodiment is also referred to as a kit of the embodiment C. Here, in a case where the chromatographic developing solution in the kit of the embodiment C includes an alkylene oxide-addition cationic surfactant and a nonionic surfactant, the kit of the embodiment C corresponds to the kit of the embodiment B. That is, the kit of the embodiment C is a concept encompassing the kit of the embodiment B.

A chromatograph device according to one or more embodiments of the present invention is a chromatograph device including a sample-receiving section, a labeling substance-retaining section, and a solid-phase support, wherein the labeling substance-retaining section includes a labeling substance bondable to an analyte, wherein the solid-phase support includes a trapping substance-retaining section containing a trapping substance bondable to the analyte, and wherein an alkylene oxide-addition cationic surfactant and a nonionic surfactant are each independently included in at least one selected from the sample-receiving section or the labeling substance-retaining section. The chromatograph device according to the embodiment is also referred to as a chromatograph device of the embodiment D.

A method for detecting an analyte contained in a sample according to one or more embodiments of the present invention is a method for detecting an analyte contained in a sample, including the following steps (1) and (2):
(1) a step of developing a mobile phase in the presence of an alkylene oxide-addition cationic surfactant and a nonionic surfactant in a solid-phase support of a chromatograph device, the mobile phase including the sample and a labeling substance bondable to an analyte; and
(2) a step of detecting the analyte in the developed mobile phase in a trapping substance-retaining section including a trapping substance bondable to the analyte contained in the solid-phase support.

The method for detecting an analyte according to the embodiment is also referred to as a method for detecting an analyte according to the embodiment E. The method for detecting an analyte according to the embodiment E is a method performable by using at least one of the chromatographic developing solution of the embodiment A, the kit of the embodiment B, the kit of the embodiment C, or the chromatograph device of the embodiment D.

Also in the kit of the embodiment B, the kit of the embodiment C, the chromatograph device of the embodiment D, and the method for detecting an analyte according to the embodiment E, the alkylene oxide-addition cationic surfactant and the nonionic surfactant are allowed to act on a labeling substance, thus achieving high-sensitivity chromatography.

The present inventor infers that, in one or more embodiments of the present invention, chromatography having high-sensitivity and excellent developability can be achieved as follows: using an alkylene oxide-addition cationic surfactant promotes the aggregation of a labeling substance or the composite of an analyte and a labeling substance, and still allows the dispersibility to be maintained owing to suitable hydrophilicity; and using a nonionic surfactant further inhibits excessive aggregation of the labeling substance or the composite of an analyte and a labeling substance. In this regard, a chromatography to which one or more embodiments of the present invention can be applied is not particularly limited, and is for example, immunochromatography and nucleic acid chromatography. At least one of immunochromatography or nucleic acid chromatography is preferable, and immunochromatography is more preferable.

### <Analyte and Specimen>

Examples of the analyte include, but are not particularly limited to, proteins, peptides, antigens, antibodies, nucleic acids (DNA, RNA, and the like), sugars (glycoproteins and glycolipids), complex carbohydrates, viruses, and bacteria. The analyte is, for example, a substance that involves verifying whether the substance is present in a specimen, in other words, a substance that involves verifying whether the substance is present in a sample.

Examples of the specimen include nasal discharge, liquid wiped off the nasal cavity, liquid wiped off the nasopharynx, liquid wiped off the pharynx, sputum, whole blood, serum, blood plasma, urine, saliva, sweat, tear, mucosa scratched off, and fecal extract. Nasal discharge, liquid wiped off the nasal cavity, liquid wiped off the nasopharynx, liquid wiped off the pharynx, or sputum, which is easy to obtain, is preferable. The specimen usually involves analyzing whether the analyte is present in the specimen.

### <Developing Solution and Sample>

The sample is a liquid component to be placed (for example, dropwise) in a chromatograph device, and developed, and is a subject to be analyzed with the chromatograph device. A sample preliminarily found containing an analyte is also referred to as a positive sample, and a sample preliminarily found containing no analyte is also referred to as a negative sample. The sample usually contains a developing solution. Examples of the sample include a sample containing a developing solution and a specimen, a sample (positive sample) containing a developing solution and an analyte, and a sample (negative sample) composed of only a developing solution.

The developing solution (chromatographic developing solution) is a liquid to be used to suitably develop a sample in a chromatograph device, and is a liquid that may be mixed with a specimen or an analyte. The developing solution may be used as a specimen diluent. Among developing solutions, a developing solution including an alkylene oxide-addition cationic surfactant and a nonionic surfactant corresponds to the above-described developing solution of the embodiment A.

The developing solution may contain a labeling substance described below in the section <Chromatograph Device>. In a case where the developing solution contains a labeling substance, a labeling substance-retaining section such as a conjugate pad can be omitted from a chromatograph device.

### <Chromatograph Device>

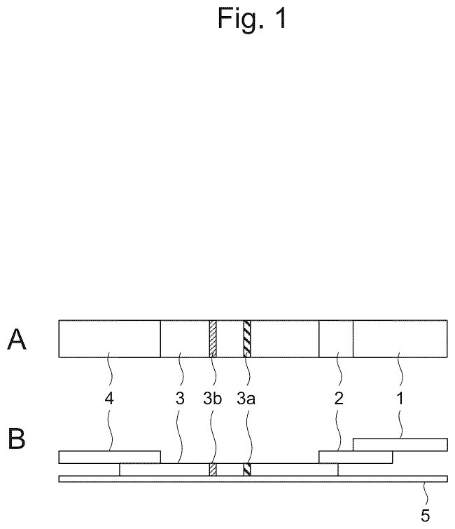

A chromatograph device to be used to perform chromatography is usually a chromatograph device including a sample-receiving section, a labeling substance-retaining section, and a solid-phase support. In this regard, the chromatograph device is also referred to as a chromatographic device. In addition, a chromatograph device for immunochromatography is also referred to as an immunochromatographic device. A chromatograph device will be described with reference to FIG. 1.

FIG. 1 (FIGS. 1A and 1B) illustrates schematic views of one example of a chromatograph device. FIG. 1A is a plan view of one example of the chromatograph device, and FIG. 1B is a cross-sectional view of one example of the chromatograph device.

A chromatograph device (test piece) illustrated in FIG. 1 has a sample-receiving section 1, a labeling substance-retaining section 2, and a solid-phase support 3, and the solid-phase support 3 has a trapping substance-retaining section 3a.

The sample-receiving section 1 is, for example, a sample pad, and is a site in which a sample is placed (for example, dropwise). Examples of the sample pad that can be used include glass fiber sample pads and cellulose fiber sample pads.

The labeling substance-retaining section 2 is, for example, a conjugate pad, and is a site containing a labeling substance bondable to an analyte. The labeling substance preferably contains colored particles. The colored particles are preferably negatively charged colored particles. Examples include metal particles, colored latex particles, colored polystyrene particles, colored cellulose particles, fluorescent cellulose particles, and silica nanoparticles including a dye. Examples of the metal particles include metal colloid particles such as gold colloid particles, silver colloid particles, and platinum colloid particles. The labeling substance has a site bondable, usually specifically bondable, to an analyte. In a case where the analyte is an antigen, the labeling substance includes, for example, an antibody (polyclonal antibody or monoclonal antibody) to the antigen. The site bondable to an analyte can be suitably set depending on the kind of the analyte. It is possible to use such a site as has been adopted in the conventional fields of immunochromatography and nucleic acid chromatography. Examples of the conjugate pad that can be used include glass fiber conjugate pads, cellulose fiber conjugate pads, polyester fiber conjugate pads, polyethylene fiber conjugate pads, and polypropylene fiber conjugate pads.

The solid-phase support 3 is, for example, a membrane, and is a site in which a mobile phase containing a sample and a labeling substance bondable to an analyte is developed. Here, in a case where the analyte is present in the sample, the mobile phase contains a composite of the analyte and the labeling substance. Examples of the membrane that can be used include nitrocellulose membranes, cellulose membranes, cellulose acetate membranes, polyethersulfone membranes, nylon membranes, polyester membranes, and glass fiber membranes.

The trapping substance-retaining section 3a included in the solid-phase support 3 is a site containing a trapping substance bondable to an analyte, and is, for example, a test line. In a case where the sample contains the analyte, a composite of the analyte and the labeling substance binds to a trapping substance in the trapping substance-retaining section 3a, thus forming a sandwich type of composite formed with three components, namely, the labeling substance, the analyte, and the trapping substance. The trapping substance may be a substance bondable to an analyte. In a case where the analyte is an antigen, for example, an antibody (polyclonal antibody or monoclonal antibody) to the antigen can be used as the trapping substance. The trapping substance can be suitably set depending on the kind of the analyte. It is possible to use such a substance as has been adopted in the conventional fields of immunochromatography and nucleic acid chromatography.

The solid-phase support 3 preferably has a control line 3b. The control line 3b is a site configured to trap a labeling substance, more specifically a labeling substance that is not included in a composite of an analyte and a labeling substance. The control line is a site containing a substance that traps a labeling substance, and is, for example, a site containing a component (antibody) capable of trapping an antibody contained in the labeling substance. The substance that traps a labeling substance can be suitably set depending on the kind of the labeling substance. It is possible to use such a substance as has been adopted in the conventional fields of immunochromatography and nucleic acid chromatography.

The chromatograph device preferably includes an absorption pad 4. The absorption pad is preferably such that, after the mobile phase developed from upstream (the sample-receiving section 1) passes through the solid-phase support 3, the mobile phase can be retained by the absorption pad so as not to flow backward. Examples of the absorption pad that can be used include cellulose fiber absorption pads, glass fiber absorption pads, and polystyrene fiber absorption pads.

The chromatograph device preferably includes a backing sheet 5. The backing sheet 5 has, for example, an adhesive layer on the surface thereof, and is fixed to the solid-phase support 3. In FIG. 1, none of the sample-receiving section 1, the labeling substance-retaining section 2, and the absorption pad 4 is fixed to the backing sheet 5, but at least one of the sample-receiving section 1, the labeling substance-retaining section 2, or the absorption pad 4 may be fixed to the backing sheet 5. In one preferable aspect, all of the sample-receiving section 1, the labeling substance-retaining section 2, and the absorption pad 4 are fixed to the backing sheet 5. Examples of the backing sheet that can be used include polypropylene backing sheets, polystyrene backing sheets, polyester backing sheets, and vinyl chloride backing sheets.

The chromatograph device (test piece) is not particularly limited to any shape or any size, and can be, for example, a generally rectangular parallelepiped having a length of 40 mm or more and 120 mm or less, a width of 3 mm or more and 20 mm or less, and a thickness of 10 μm or more and 5.0 mm or less. The shape and size can be suitably changed depending on the kind of the analyte, the number of the trapping substance-retaining sections, and the like.

The chromatograph device can include a housing case (not shown) for containing a test piece (that includes the sample-receiving section 1, the labeling substance-retaining section 2, and the solid-phase support 3, and preferably includes the absorption pad 4 and the backing sheet 5). The housing case is composed of, for example, a water-impermeable and moldable material such as polyethylene, polypropylene, polystyrene, polyethylene terephthalate, or polyvinyl chloride, and has such a form as to cover the whole test piece, and be provided with openings or windows at the positions corresponding to the sample-receiving section 1 and the trapping substance-retaining section 3a, preferably further at the position corresponding to the control line 3b. Being provided with a housing can prevent a mobile phase developed from being leaked out.

### <Alkylene Oxide-Addition Cationic Surfactant>

In one or more embodiments of the present invention, an alkylene oxide-addition cationic surfactant is used. The alkylene oxide included in the alkylene oxide-addition cationic surfactant is, for example, ethylene oxide or propylene oxide, and is preferably ethylene oxide. That is, the alkylene oxide-addition cationic surfactant preferably includes an ethylene oxide-addition cationic surfactant. As the alkylene oxide-addition cationic surfactant, one kind of or two or more kinds of such surfactant(s) may be used.

In one or more embodiments of the present invention, the alkylene oxide-addition cationic surfactant includes at least one surfactant selected from a surfactant represented by the following general formula (I) or a surfactant represented by the following general formula (II):

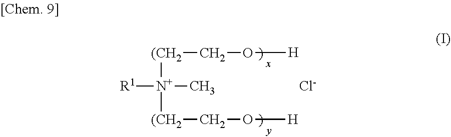

(wherein, in the general formula (I), R¹ is a C₁₋₃₀ saturated or unsaturated hydrocarbon group, x and y are each independently an integer of 1 to 49, and x+y is 2 to 50.)

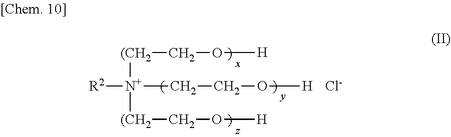

(wherein, in the general formula (II), R² is a C₁₋₃₀ saturated or unsaturated hydrocarbon group, x, y, and z are each independently an integer of 1 to 48, and x+y+z is 3 to 50.)

R¹ and R² above are each independently a C₁₋₃₀ saturated or unsaturated hydrocarbon group, preferably a C₄₋₂₆ saturated or unsaturated hydrocarbon group, more preferably a C₆₋₂₄ saturated or unsaturated hydrocarbon group, particularly preferably a C₈₋₂₂ saturated or unsaturated hydrocarbon group. In a case where the C₁₋₃₀ saturated or unsaturated hydrocarbon group is an unsaturated hydrocarbon group, the degree of unsaturation is preferably 1 to 3, more preferably 1. The C₁₋₃₀ saturated or unsaturated hydrocarbon group is preferably a saturated hydrocarbon group or an unsaturated hydrocarbon group having a degree of unsaturation of 1, more preferably a saturated hydrocarbon group. For the C₁₋₃₀ saturated or unsaturated hydrocarbon group, being a C₁₋₃₀ saturated hydrocarbon group has the same meaning as being a C₁₋₃₀ alkyl group. The C₁₋₃₀ saturated or unsaturated hydrocarbon group may be a linear hydrocarbon group, a branched hydrocarbon group, or a hydrocarbon group having a ring structure, preferably a linear hydrocarbon group or a branched hydrocarbon group, more preferably a linear hydrocarbon group.

In the general formula (I), x and y are each independently an integer of 1 to 49, preferably an integer of 1 to 34, more preferably an integer of 1 to 24, particularly preferably an integer of 1 to 14. In the general formula (I), x+y is 2 to 50, preferably 2 to 35, more preferably 2 to 25, particularly preferably 2 to 15.

In the general formula (II), x, y, and z are each independently an integer of 1 to 48, preferably an integer of 1 to 33, more preferably an integer of 1 to 23, particularly preferably an integer of 1 to 13. In the general formula (II), x+y+z is 3 to 50, preferably 3 to 35, more preferably 3 to 25, particularly preferably 3 to 15.

In one or more embodiments of the present invention, the alkylene oxide-addition cationic surfactant includes at least one surfactant selected from PEG-5 stearyl ammonium chloride, PEG-2 oleammonium chloride, PEG-2 cocomonium chloride, PEG-15 cocomonium chloride, or PEG-15 steamonium chloride.

The HLB value of the alkylene oxide-addition cationic surfactant is preferably 22 or more and 31 or less, more preferably 22.5 or more and 29 or less, particularly preferably 22.5 or more and 28 or less. The HLB value of the alkylene oxide-addition cationic surfactant is usually an HLB value determined by the Davies method. In the Davies method, the HLB value can be determined in accordance with the following formula. In this regard, the HLB value of a surfactant mixture can be determined as the weighted average of the HLB values of the respective components.

$$HLB value ( the Davies method ) = 7 + the sum of the number of hydrophilic groups - the sum of the number of lipophilic groups$$

For the number of groups to be used for the Davies method, reference is made to O Boen Ho, J. Colloid Interface Sci., 198, 249-260 (1998) and A.N.S.C. LLC, “Surface Chemistry HLB & Emulsification,” AkzoNobel Surf. Chem., 1-15 (2008).

As the alkylene oxide-addition cationic surfactant, a commercially available product may be used. Examples of the commercially available product that can be used include: CATINAL (registered trademark; hereinafter, the phrase “registered trademark” is omitted in some cases) SPC-20V-S(tri(polyoxyethylene)stearyl ammonium chloride (5 E.O.), PEG-5 stearyl ammonium chloride; the HLB value, 25.4 (the Davies method); manufactured by Toho Chemical Industry Co., Ltd.); LIPOTHOQUAD (registered trademark; hereinafter, the phrase “registered trademark” is omitted in some cases) C/12 (ETHOQUAD (ETHOQUAD (registered trademark; hereinafter, the phrase “registered trademark” is omitted in some cases)) C/12) (cocoalkylbis(2-hydroxyethyl)methylammonium chloride, PEG-2 cocomonium chloride; the HLB value, 25.8 (the Davies method), manufactured by Lion Specialty Chemicals Co., Ltd. or Nouryon N.V.); LIPOTHOQUAD O/12 (ETHOQUAD O/12) (oleylbis(2-hydroxyethyl)methylammonium chloride, PEG-2 oleammonium chloride; the HLB value, 23.4 (the Davies method), manufactured by Lion Specialty Chemicals Co., Ltd. or Nouryon N.V.); ETHOQUAD 18/25 (polyoxyethyleneoctadecylmethylammonium chloride (15 E.O.), PEG-15 steamonium chloride; the HLB value, 28 (the Davies method), manufactured by Nouryon N.V.); and LIPOTHOQUAD C/25 (ETHOQUAD C/25) (polyoxyethylenecocoalkylmethylammonium chloride (15 E.O.), PEG-15 cocomonium chloride; the HLB value, 30.4 (the Davies method), manufactured by Lion Specialty Chemicals Co., Ltd. or Nouryon N.V.).

### <Nonionic Surfactant>

In one or more embodiments of the present invention, a nonionic surfactant is used. As the nonionic surfactant, one kind of or two or more kinds of such surfactant(s) may be used.

In one or more embodiments of the present invention, the nonionic surfactant includes at least one surfactant selected from a surfactant represented by the following general formula (III), a surfactant represented by the following general formula (IV), a surfactant represented by the following general formula (V), a surfactant represented by the following general formula (VI), a surfactant represented by the following general formula (VII), or a surfactant represented by the following general formula (VIII).

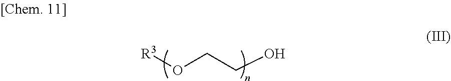

(wherein, in the general formula (III), R³ is a C₁₋₃₀ saturated or unsaturated hydrocarbon group, and n is an integer of 1 to 50.)

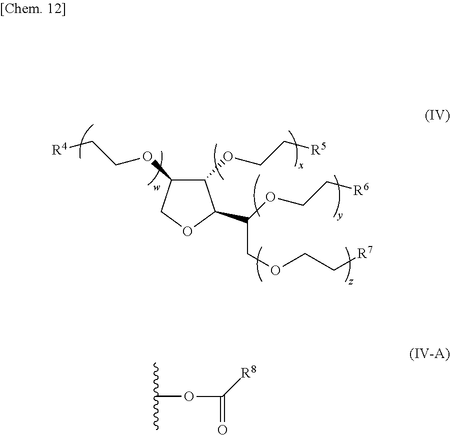

(wherein, in the general formula (IV), R⁴ to R⁷ are each independently a group represented by the above-described general formula (IV-A) or a hydroxy group; at least one of R⁴ to R⁷ is a group represented by the above-described general formula (IV-A); at least one of R⁴ to R⁷ is a hydroxy group;
w, x, y, and z are each independently an integer of 1 to 47, and w+x+y+z is 4 to 50; and
   in the general formula (IV-A), R⁸ is a C₁₋₃₀ saturated or unsaturated hydrocarbon group; and the wavy line represents a binding site to a carbon atom.)

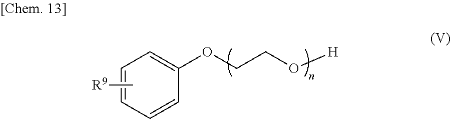

(wherein, in the general formula (V), R⁹ is a C₁₋₃₀ saturated or unsaturated hydrocarbon group, and n is an integer of 1 to 50.)

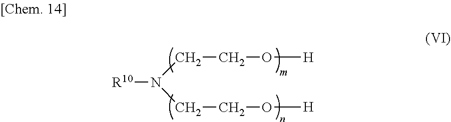

(wherein, in the general formula (VI), R¹⁰ is a C₁₋₃₀ saturated or unsaturated hydrocarbon group, m and n are each independently an integer of 1 to 49, and m+n is 2 to 50.)

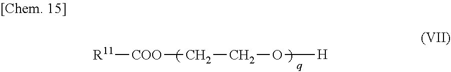

(wherein, in the general formula (VII), R¹¹ is a C₁₋₃₀ saturated or unsaturated hydrocarbon group, and q is an integer of 1 to 300.)

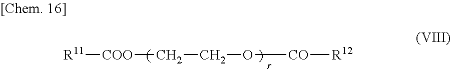

(wherein, in the general formula (VIII), R¹¹ and R¹² are each independently a C₁₋₃₀ saturated or unsaturated hydrocarbon group, and r is an integer of 1 to 300.)

R³, R⁸, R⁹, R¹⁰, R¹¹, and R¹² above are each independently a C₁₋₃₀ saturated or unsaturated hydrocarbon group, preferably a C₄₋₂₆ saturated or unsaturated hydrocarbon group, more preferably a C₆₋₂₄ saturated or unsaturated hydrocarbon group, particularly preferably a C₈₋₂₂ saturated or unsaturated hydrocarbon group. In a case where the C₁₋₃₀ saturated or unsaturated hydrocarbon group is an unsaturated hydrocarbon group, the degree of unsaturation is preferably 1 to 3, more preferably 1. The C₁₋₃₀ saturated or unsaturated hydrocarbon group is preferably a saturated hydrocarbon group or an unsaturated hydrocarbon group having a degree of unsaturation of 1, more preferably a saturated hydrocarbon group. For the C₁₋₃₀ saturated or unsaturated hydrocarbon group, having a C₁₋₃₀ saturated hydrocarbon group has the same meaning as having a C₁₋₃₀ alkyl group. The C₁₋₃₀ saturated or unsaturated hydrocarbon group may be a linear hydrocarbon group, a branched hydrocarbon group, or a hydrocarbon group having a ring structure, preferably a linear hydrocarbon group or a branched hydrocarbon group.

In the general formula (III), n is an integer of 1 to 50, preferably an integer of 2 to 49, more preferably an integer of 3 to 48.

In the general formula (IV), R⁴ to R⁷ are each independently a group represented by the above-described general formula (IV-A) or a hydroxy group; at least one of R⁴ to R⁷ is a group represented by the above-described general formula (IV-A); at least one of R⁴ to R⁷ is a hydroxy group. One of R⁴ to R⁷ is a group represented by the above-described general formula (IV-A). In one preferable aspect, three of R⁴ to R⁷ are hydroxy groups.

In the general formula (IV), w, x, y, and z are each independently an integer of 1 to 47, preferably an integer of 1 to 32, more preferably an integer of 1 to 22, particularly preferably an integer of 1 to 17. In the general formula (IV), w+x+y+z is 4 to 50, preferably 4 to 35, more preferably from 4 to 25, particularly preferably 4 to 20.

In the general formula (V), n is an integer of 1 to 50, preferably an integer of 3 to 40, more preferably an integer of 5 to 30.

In the general formula (VI), m and n are each independently an integer of 1 to 49, preferably an integer of 2 to 46, more preferably an integer of 3 to 41, particularly preferably an integer of 5 to 35. In the general formula (VI), m+n is 2 to 50, preferably 4 to 48, more preferably from 6 to 44, particularly preferably 10 to 40. In addition, n=m is one preferable aspect.

In the general formula (VII), q is an integer of 1 to 300, preferably an integer of 2 to 100, more preferably an integer of 3 to 50.

In the general formula (VIII), r is an integer of 1 to 300, preferably an integer of 2 to 100, more preferably an integer of 3 to 50.

In one or more embodiments of the present invention, the nonionic surfactant includes at least one surfactant selected from a polyoxyethylenealkyl ether, polyoxyethylenesorbitan fatty acid ester, polyoxyethylenealkylphenyl ether, polyoxyethylenealkyl amine, or polyethylene glycol fatty acid ester.

The HLB value of the nonionic surfactant is preferably 10 or more and 19 or less, more preferably 10.5 or more and 18.5 or less. The HLB value of the nonionic surfactant is usually an HLB value determined by the Griffin method. In the Griffin method, the HLB value can be determined in accordance with the following formula. In this regard, the HLB value of a surfactant mixture can be determined as the weighted average of the HLB values of the respective components.

$$HLB ( the Griffin method ) = 20 \times the sum of the formula weights of hydrophilic moieties / the molecular weight of a surfactant$$

As the nonionic surfactant, a commercially available product may be used. Examples of the commercially available product that can be used include: Triton (registered trademark; hereinafter, the phrase “registered trademark” is omitted in some cases) X-100 (polyoxyethyleneoctylphenyl ether (9.5 E.O.); the HLB value, 13.4 (the Griffin method); manufactured by Sigma-Aldrich); Tween (registered trademark; hereinafter, the phrase “registered trademark” is omitted in some cases) 20 (polyoxyethylenesorbitan monolaurate (20 E.O.), polysorbate 20; the HLB value, 16.7 (the Griffin method); manufactured by Sigma-Aldrich); EMULGEN (registered trademark; hereinafter, the phrase “registered trademark” is omitted in some cases) 150 (polyoxyethylenelauryl ether (47 E.O.), polyoxyethylenedodecyl ether (47 E.O.); the HLB value, 18.4 (the Griffin method); manufactured by Kao Corporation); EMULGEN 108 (polyoxyethylenelauryl ether (6 E.O.), polyoxyethylenedodecyl ether (6 E.O.); the HLB value, 12.1 (the Griffin method); manufactured by Kao Corporation); Brij (registered trademark; hereinafter, the phrase “registered trademark” is omitted in some cases) 35 (polyoxyethylenelauryl ether (23 E.O.), polyoxyethylenedodecyl ether (23 E.O.); the HLB value, 16.9 (the Griffin method); manufactured by Sigma-Aldrich); AMIET (registered trademark; hereinafter, the phrase “registered trademark” is omitted in some cases) 105A (polyoxyethylene coconut alkyl amine (5 E.O.), polyoxyethylene coco alkyl amine (5 E.O.); the HLB value, 10.8 (the Griffin method); manufactured by Kao Corporation); AMIET 320 (polyoxyethylene hydrogenated tallow amine (20 E.O.), polyoxyethylene hydrogenated tallow amine (20 E.O.); the HLB value, 15.4 (the Griffin method); manufactured by Kao Corporation); Tween 40 (polyoxyethylenesorbitan monopalmitate (20 E.O.); the HLB value, 15.6 (the Griffin method); manufactured by Sigma-Aldrich); Tween 60 (polyoxyethylenesorbitan monostearate (20 E.O.); the HLB value, 14.9 (the Griffin method); manufactured by Sigma-Aldrich); Tween 80 (polyoxyethylenesorbitan monooleate (20 E.O.); the HLB value, 15.0 (the Griffin method); manufactured by Sigma-Aldrich); Tween 65 (polyoxyethylenesorbitan tristearate (20 E.O.); the HLB value, 10.5 (the Griffin method); manufactured by Sigma-Aldrich); Tween 85 (polyoxyethylenesorbitan trioleate (20 E.O.); the HLB value, 11.0 (the Griffin method); manufactured by Sigma-Aldrich); Nonidet (registered trademark; hereinafter, the phrase “registered trademark” is omitted in some cases) P-40 (octylphenol ethoxylate (9 E.O.); the HLB value, 13.1 (the Griffin method); manufactured by Sigma-Aldrich); Triton X-102 (octylphenol ethoxylate (12 E.O.); the HLB value, 14.6 (the Griffin method); manufactured by Sigma-Aldrich); Triton X-114 (octylphenol ethoxylate (7-8 E.O.); the HLB value, 12.4 (the Griffin method); manufactured by Sigma-Aldrich); Triton X-165 (octylphenol ethoxylate (15-16 E.O.); the HLB value, 15.8 (the Griffin method); manufactured by Sigma-Aldrich); Triton X-405 (octylphenol ethoxylate (40 E.O.); the HLB value, 17.9 (the Griffin method); manufactured by Sigma-Aldrich); Triton N-101 (nonylphenol ethoxylate (9-10 E.O.); the HLB value, 13.5 (the Griffin method); manufactured by Sigma-Aldrich).

### Chromatographic Developing Solution (Embodiment A)

The chromatographic developing solution of the embodiment A includes the above-described alkylene oxide-addition cationic surfactant and the above-described nonionic surfactant. In one or more embodiments of the present invention, the chromatographic developing solution of the embodiment A is a specimen diluent.

In the chromatographic developing solution of the embodiment A, the nonionic surfactant/the alkylene oxide-addition cationic surfactant (as a weight ratio), which is the ratio of the amount of the nonionic surfactant to the amount of the alkylene oxide-addition cationic surfactant, is preferably 0.14 or more and 40 or less, more preferably 0.15 or more and 30 or less, particularly preferably 0.16 or more and 20 or less.

The chromatographic developing solution of the embodiment A preferably contains the alkylene oxide-addition cationic surfactant at 0.05 wt % or more and 14 wt % or less, more preferably 0.07 wt % or more and 13 wt % or less, particularly preferably 0.1 wt % or more and 12.5 wt % or less.

The chromatographic developing solution of the embodiment A preferably contains the nonionic surfactant at 0.6 wt % or more and 12 wt % or less, more preferably 0.65 wt % or more and 11 wt % or less, particularly preferably 0.75 wt % or more and 10 wt % or less.

The chromatographic developing solution of the embodiment A usually contains water as a solvent, and may include a component other than the alkylene oxide-addition cationic surfactant and the nonionic surfactant. The component other than the alkylene oxide-addition cationic surfactant and the nonionic surfactant is, for example, a component to be used for a conventional chromatographic developing solution.

Examples of the component other than the alkylene oxide-addition cationic surfactant and the nonionic surfactant include buffers, stabilizing components, and preservative components. Examples of the buffer include a tris buffer, phosphate buffer, citrate buffer, Veronal buffer, borate buffer, and Good's buffer. Examples of the stabilizing component include; polymer compounds such as MPC (2-methacryloyloxyethylphosphorylcholine) polymer, polyethylene glycol, polyvinylpyrrolidone, polyvinyl alcohol, and poly(2-ethyl-2-oxazoline); albumins such as bovine serum albumins; globulins; caseins; serums; water-soluble gelatins; surfactants; sugars; polysaccharides; and chelators. In addition, examples of the preservative component include salicylic acid, benzoic acid, and sodium azide.

In one or more embodiments of the present invention, the chromatographic developing solution of the embodiment A further includes a nonspecific-adsorption inhibitor. Examples of the nonspecific-adsorption inhibitor include; L-arginine hydrochloride; ethylenediaminetetraacetic acid (EDTA) and salts of EDTA, such as EDTA-2Na; organic acids such as succinic acid, malic acid, tartaric acid, and citrate, and salts thereof; and inorganic salts such as sodium chloride, lithium chloride, potassium chloride, and magnesium chloride. Using the chromatographic developing solution containing a nonspecific-adsorption inhibitor inhibits the coloration due to a nonspecific reaction in the background (portion other than a test line and a control line in the membrane), thus making it possible to enhance the visibility of the coloration of the test line and the control line.

In one or more embodiments of the present invention, the chromatographic developing solution of the embodiment A further includes a dispersibility improver. Examples of the dispersibility improver include ampholytic surfactants and water-miscible organic solvents. Examples of the ampholytic surfactant include: sulfobetaine ampholytic surfactants such as CHAPS (3-[(3-cholamido-propyl)dimethylammonio]-1-propanesulfonate), CHAPSO (3-[(3-cholamido-propyl)dimethylammonio]-2-hydroxy-1-propanesulfonate), sulfobetaine-8 (3-(dimethyloctylammonio)propanesulfonate), sulfobetaine-10 (3-(decyldimethylammonio)propanesulfonate), sulfobetaine-12 (3-(dodecyldimethylammonio)propanesulfonate), sulfobetaine-14 (3-(myristyldimethylammonio)propanesulfonate), sulfobetaine-16 (3-(palmityldimethylammonio)propanesulfonate), and sulfobetaine-18 (3-(stearyldimethylammonio)propanesulfonate); amidobetain ampholytic surfactants such as palm acid amidopropyl betaine, amidopropylbetaine laurate, lauryldimethylaminoacetic acid betaine, laurylbetaine, and cocobetaine; and lecithin derived from soya bean or egg. Examples of the water-miscible organic solvent include ethanol, methanol, isopropanol, DMSO (dimethyl sulfoxide), DMF (N,N-dimethylformamide), and NMP (N-methyl-2-pyrrolidone). Using the chromatographic developing solution containing a dispersibility improver enhances the dispersion stability during the development of a labeling substance, and thereby, further enhances the developability of the developing solution, thus inhibiting the residual coloration of the labeling substance in the background (portion other than a test line and a control line in the membrane), and enabling a decrease in the risk of erroneous determination about false positiveness due to the background coloration that looks like a test line. In particular, even a chromatograph device that has passed a long time since the production of the product can enhance the dispersion stability during the development of the labeling substance to enhance the developability.

In one or more embodiments of the present invention, the chromatographic developing solution of the embodiment A further includes an interference inhibitor. It is known that interference factors such as a heterophilic antibody (HA) and a rheumatoid factor (RF) that are contained in a specimen in some cases can cause false positiveness or false negativeness in immunochromatography. Examples of the heterophilic antibody include a human anti-mouse antibody (HAMA), a human anti-goat antibody (HAGA), a human anti-sheep antibody (HASA), and a human anti-rabbit antibody (HARA). Using the chromatographic developing solution containing an interference inhibitor can decrease the risk of erroneous determination about false positiveness or false negativeness due to the influence of an interference factor. Examples of the interference inhibitor include: an antibody to the interference factor present in a specimen; an immunoglobulin the producing-animal species of which is the same as or different from the producing-animal species of an antibody to be used as a trapping substance or a labeling substance in immunoassay; HBR1 (manufactured by Scantibodies Laboratory, Inc.); and TRU Block (manufactured by Meridian Life Science Inc.).

### Kits (Embodiment B and Embodiment C)

The kit of the embodiment B is a kit for detecting an analyte, including: the chromatographic developing solution of the embodiment A; and a chromatograph device including a sample-receiving section, a labeling substance-retaining section, and a solid-phase support, wherein the labeling substance-retaining section includes a labeling substance bondable to an analyte, and wherein the solid-phase support includes a trapping substance-retaining section containing a trapping substance bondable to the analyte. The kit of the embodiment B is one aspect of utilization of the chromatographic developing solution of the embodiment A.

In addition, the kit of the embodiment C is, for example, a kit for detecting an analyte, comprising: a chromatographic developing solution; and a chromatograph device including a sample-receiving section, a labeling substance-retaining section, and a solid-phase support, wherein the labeling substance-retaining section includes a labeling substance bondable to an analyte, wherein the solid-phase support includes a trapping substance-retaining section containing a trapping substance bondable to the analyte, and wherein the alkylene oxide-addition cationic surfactant and the nonionic surfactant are each independently included in at least one selected from the chromatographic developing solution, the sample-receiving section, or the labeling substance-retaining section.

In the kit of the embodiment C, the alkylene oxide-addition cationic surfactant and the nonionic surfactant are each independently included in at least one selected from the chromatographic developing solution, the sample-receiving section, or the labeling substance-retaining section. For example, both of the alkylene oxide-addition cationic surfactant and the nonionic surfactant may be included in the chromatographic developing solution, may be included in the sample-receiving section, or may be included in the labeling substance-retaining section. In another example, it is also possible that the nonionic surfactant is included in the chromatographic developing solution, and that the alkylene oxide-addition cationic surfactant is included in at least one of the sample-receiving section or the labeling substance-retaining section. The alkylene oxide-addition cationic surfactant may be included in two or three selected from the chromatographic developing solution, the sample-receiving section, and the labeling substance-retaining section. In addition, the nonionic surfactant may be included in two or three selected from the chromatographic developing solution, the sample-receiving section, and the labeling substance-retaining section.

In one preferable aspect of the kit of the embodiment C, the chromatographic developing solution includes the nonionic surfactant, and the labeling substance-retaining section includes the alkylene oxide-addition cationic surfactant and the nonionic surfactant.

The kit of the embodiment B includes the chromatographic developing solution of the embodiment A, and thus, includes the developing solution, the alkylene oxide-addition cationic surfactant, and the nonionic surfactant, but other sections, for example, the sample-receiving section and the labeling substance-retaining section may further include at least one of the alkylene oxide-addition cationic surfactant or the nonionic surfactant.

In any of the kit of the embodiment B and the kit of the embodiment C, the alkylene oxide-addition cationic surfactant and the nonionic surfactant can act on the labeling substance before the sample is developed in the solid-phase support, or while the sample is developed in the solid-phase support, and thus, the kits can exhibit high-sensitivity (high color-developing capability) and excellent developability.

In the kit of the embodiment B and the kit of the embodiment C, the total nonionic surfactant/the total alkylene oxide-addition cationic surfactant (as a weight ratio), which is the ratio of the total amount of the nonionic surfactant to the total amount of the alkylene oxide-addition cationic surfactant, is preferably 0.14 or more and 40 or less, more preferably 0.15 or more and 30 or less, particularly preferably 0.16 or more and 20 or less, wherein the nonionic surfactant and the cationic surfactant are contained in the chromatographic developing solution developed in the sample-receiving section, contained in the sample-receiving section, and contained in the labeling substance-retaining section.

In any one of the kit of the embodiment B and the kit of the embodiment C, at least one component selected from the nonspecific-adsorption inhibitor, the dispersibility improver, or the interference inhibitor may be each independently included in at least one selected from the chromatographic developing solution, the sample-receiving section, or the labeling substance-retaining section. For example, the kit may include one component, two components, or three components of at least one component selected from the nonspecific-adsorption inhibitor, the dispersibility improver, or the interference inhibitor. In addition, in a case where the kit includes two or more of the at least one component selected from the nonspecific-adsorption inhibitor, the dispersibility improver, or the interference inhibitor, the two or more components may be included in the same section or different sections of the chromatographic developing solution, the sample-receiving section, and the labeling substance-retaining section. In addition, a specific component may be included in a plurality of sections.

### Chromatograph Device (Embodiment D)

A chromatograph device the embodiment D is a chromatograph device including a sample-receiving section, a labeling substance-retaining section, and a solid-phase support, wherein the labeling substance-retaining section includes a labeling substance bondable to an analyte, wherein the solid-phase support includes a trapping substance-retaining section containing a trapping substance bondable to the analyte, and wherein the alkylene oxide-addition cationic surfactant and the nonionic surfactant are each independently included in at least one selected from the sample-receiving section or the labeling substance-retaining section.

In the chromatograph device of the embodiment D, the alkylene oxide-addition cationic surfactant and the nonionic surfactant are each independently included in at least one selected from the sample-receiving section or the labeling substance-retaining section. For example, both of the alkylene oxide-addition cationic surfactant and the nonionic surfactant may be included in the sample-receiving section, or may be included in the labeling substance-retaining section. In another example, it is also possible that the nonionic surfactant is included in the sample-receiving section, and that the alkylene oxide-addition cationic surfactant is included in the labeling substance-retaining section. The alkylene oxide-addition cationic surfactant may be included in both of the sample-receiving section and the labeling substance-retaining section. In addition, the nonionic surfactant may be included in both of the sample-receiving section and the labeling substance-retaining section.

The chromatograph device of the embodiment D includes the alkylene oxide-addition cationic surfactant and the nonionic surfactant in at least one selected from the sample-receiving section of the labeling substance-retaining section. Thus, even if a conventional developing solution is used as a chromatographic developing solution, the alkylene oxide-addition cationic surfactant and the nonionic surfactant can act on the labeling substance while the sample is developed in the solid-phase support, and thus, the device can exhibit high-sensitivity (high color-developing capability) and excellent developability.

In the chromatograph device of the embodiment D, the total nonionic surfactant/the total alkylene oxide-addition cationic surfactant (as a weight ratio), which is the ratio of the total amount of the nonionic surfactant to the total amount of the alkylene oxide-addition cationic surfactant, is preferably 0.14 or more and 40 or less, more preferably 0.15 or more and 30 or less, particularly preferably 0.16 or more and 20 or less, wherein the nonionic surfactant and the cationic surfactant are contained in the sample-receiving section and the labeling substance-retaining section.

At least one component selected from the nonspecific-adsorption inhibitor, the dispersibility improver, or the interference inhibitor may be each independently included in at least one selected from the sample-receiving section or the labeling substance-retaining section. For example, the chromatograph device may include one component, two components, or three components of at least one component selected from the nonspecific-adsorption inhibitor, the dispersibility improver, or the interference inhibitor. In addition, in a case where the chromatograph device includes two or more of the at least one component selected from the nonspecific-adsorption inhibitor, the dispersibility improver, or the interference inhibitor, the two or more components may be included in the same section or different sections of the sample-receiving section and the labeling substance-retaining section. In addition, a specific component may be included in a plurality of sections.

### Method for Detecting Analyte Contained in Sample (Embodiment E)

A method for detecting an analyte contained in a sample according to the embodiment E is a method for detecting an analyte contained in a sample, including the following steps (1) and (2):
(1) a step of developing a mobile phase in the presence of the alkylene oxide-addition cationic surfactant and the nonionic surfactant in a solid-phase support of a chromatograph device, the mobile phase including the sample and a labeling substance bondable to an analyte; and
(2) a step of detecting the analyte in the developed mobile phase in a trapping substance-retaining section including a trapping substance bondable to the analyte contained in the solid-phase support.

The method for detecting an analyte according to the embodiment E is a method performable by using at least one of the chromatographic developing solution of the embodiment A, the kit of the embodiment B, the kit of the embodiment C, or the chromatograph device of the embodiment D.

That is, the alkylene oxide-addition cationic surfactant and the nonionic surfactant that are present in the mobile phase may be included in the chromatographic developing solution, may be included in the sample-receiving section, or may be included in the labeling substance-retaining section.

### EXAMPLES

One or more embodiments of the present invention will be described below with reference to Examples, but the present disclosure is not limited to these Examples.

In Examples and Comparative Examples, the following reagents were used.

### Nonionic Surfactant:

Triton X-100 (polyoxyethyleneoctylphenyl ether (9.5 E.O.), having an HLB value of 13.4 (the Griffin method, cited from documents of The Dow Chemical Company), manufactured by Sigma-Aldrich);
Tween 20 (polyoxyethylenesorbitan monolaurate (20 E.O.), having an HLB value of 16.7 (the Griffin method, cited from documents of the manufacturer), manufactured by Sigma-Aldrich); EMULGEN 150 (polyoxyethylenelauryl ether (47 E.O.) having an HLB value of 18.4 (the Griffin method, cited from documents of the manufacturer), manufactured by Kao Corporation);
EMULGEN 108 (polyoxyethylenelauryl ether (6 E.O.), having an HLB value of 12.1 (the Griffin method, cited from documents of the manufacturer), manufactured by Kao Corporation);
Brij 35 (polyoxyethylenelauryl ether (23 E.O.), having an HLB value of 16.9 (the Griffin method, cited from documents of the manufacturer), manufactured by Sigma-Aldrich);
AMIET 105A (polyoxyethylene coconut alkyl amine (5 E.O.), having an HLB value of 10.8 (the Griffin method, cited from documents of the manufacturer), manufactured by Kao Corporation); and
AMIET 320 (polyoxyethylene hydrogenated tallow amine (20 E.O.), having an HLB value of 15.4 (the Griffin method, cited from documents of the manufacturer), manufactured by Kao Corporation)

### Cationic Surfactant:

CATINAL SPC-20V-S(tri(polyoxyethylene)stearyl ammonium chloride (5 E.O.) (20 wt %), having an HLB value of 25.4 (the Davies method), manufactured by Toho Chemical Industry Co., Ltd.);
LIPOTHOQUAD C/12 (cocoalkylbis(2-hydroxyethyl)methylammonium chloride (70 to 80 wt %), having an HLB value of 25.8 (the Davies method, cited from documents of Akzo Nobel N.V. (now Nouryon N.V.)), manufactured by Lion Specialty Chemicals Co., Ltd.);
LIPOTHOQUAD O/12 (oleylbis(2-hydroxyethyl)methylammonium chloride (70 to 80 wt %), having an HLB value of 23.4 (the Davies method, cited from documents of Akzo Nobel N.V. (now Nouryon N.V.)), manufactured by Lion Specialty Chemicals Co., Ltd.);
CTAC (cetyltrimethylammonium chloride, having an HLB value of 21.4 (the Davies method), manufactured by Sigma-Aldrich); and
STAC (stearyltrimethylammonium chloride, having an HLB value of 20.5 (the Davies method), manufactured by Fujifilm Wako Pure Chemical Corporation)

### Anionic Surfactant:

SDS (sodium dodecylsulfate), manufactured by Fujifilm Wako Pure Chemical Corporation; and
SDBS (sodium dodecylbenzenesulfonate), manufactured by Kanto Chemical Co., Inc.

### Ampholytic Surfactant:

CHAPS (3-[(3-cholamido-propyl)dimethylammonio]-1-propanesulfonate), manufactured by Dojindo Laboratories;
Sulfobetaine-14 (3-(myristyldimethylammonio)propanesulfonate), manufactured by Tokyo Chemical Industry Co., Ltd.;
Lecithin (derived from soya bean), manufactured by Fujifilm Wako Pure Chemical Corporation; and
AMPHITOL 20AB (amidopropylbetaine laurate (30 wt %)), manufactured by Kao Corporation

### Additive:

MPC (2-methacryloyloxyethylphosphorylcholine) polymer, N102, manufactured by NOF Corporation;
Polyethylene glycol (having an average molecular weight of 4,000), manufactured by Fujifilm Wako Pure Chemical Corporation;
Polyvinylpyrrolidone (having an average molecular weight of 40,000), manufactured by Fujifilm Wako Pure Chemical Corporation;
Polyvinyl alcohol (having a degree of polymerization of 500), manufactured by Fujifilm Wako Pure Chemical Corporation;
Poly(2-ethyl-2-oxazoline) (having an average molecular weight of 50,000), manufactured by Sigma-Aldrich;
Dextran (having an average molecular weight of 40,000), manufactured by Fujifilm Wako Pure Chemical Corporation;
Bovine serum albumin, manufactured by Fujifilm Wako Pure Chemical Corporation;
Casein, manufactured by Fujifilm Wako Pure Chemical Corporation;
DMSO (dimethyl sulfoxide), manufactured by Fujifilm Wako Pure Chemical Corporation;
DMF (N,N-dimethylformamide), manufactured by Fujifilm Wako Pure Chemical Corporation; and
Mouse IgM, manufactured by Institute of Immunology Co., Ltd.

### Example 1

### (1) Production of Anti-SARS-COV-2 NP Antibody-Bound Gold Colloid

With 9 mL of a gold colloid solution (having a particle diameter of 40 nm and a concentration of 9.0×10¹⁰ [particles/mL]; manufactured by BBI Solutions Group Limited) adjusted to a pH of 8.0 with a 5 mM phosphate buffer, 1 mL of an aqueous solution of a 100 μg/mL mouse anti-SARS-COV-2 NP monoclonal antibody was mixed. The resulting mixture was incubated at room temperature for 15 minutes.

With the mixture, 0.5 mL of an aqueous solution of 1 wt % polyethylene glycol (having an average molecular weight of 20,000) and 1 mL of an aqueous solution of 10 wt % bovine serum albumin were further mixed, and then, the resulting mixture was again incubated at room temperature overnight.

The mixture incubated was centrifuged at room temperature at 7,000×g for 15 minutes, the supernatant was removed, and then, a 5 mM phosphate buffer having a pH of 7.0 was added to the resulting precipitate, which was re-suspended. Buffer replacement was performed again using a phosphate buffer to obtain a suspension of an anti-SARS-COV-2 NP antibody-bound gold colloid (an antibody-bound gold colloid suspension).

### (2) Production of Anti-SARS-COV-2 NP Antibody-Bound Gold Colloid-Coated Conjugate Pad

To the antibody-bound gold colloid suspension produced in (1) above, sucrose, polyethylene glycol (having an average molecular weight of 20,000), and bovine serum albumin were added at 5 wt %, 0.05 wt %, and 1 wt % respectively to prepare an antibody-bound gold colloid coating liquid.

The antibody-bound gold colloid coating liquid was uniformly applied at 0.5 μL/mm² to a glass fiber pad cut out in a shape 7 mm high and 300 mm long.

Then, the glass fiber pad coated with the antibody-bound gold colloid coating liquid was dried with a vacuum dryer to obtain a conjugate pad.

### (3) Production of Anti-SARS-COV-2 NP Antibody-Coated Membrane

To a 10 mm high position of a nitrocellulose membrane (HF120, manufactured by Merk Millipore) cut out in a shape 25 mm high and 300 mm long, a solution containing a 1 mg/ml mouse anti-SARS-COV-2 NP monoclonal antibody and 2.5 wt % sucrose in a 5 mM phosphate buffer was applied, using a dispenser, at 1 μL/cm in the form of a line 1 mm wide and perpendicular to the direction of development to produce a test line.

To a 16 mm high position of the nitrocellulose membrane, a solution containing 1 mg/mL goat anti-mouse immunoglobulin polyclonal antibody and 2.5 wt % sucrose in a 5 mM phosphate buffer was applied, using a dispenser, at 1 μL/cm in the form of a line 1 mm wide and perpendicular to the direction of development to produce a control line.

Then, the nitrocellulose membrane with the test line and the control line produced by coating was dried using a vacuum dryer to obtain an antibody-coated membrane.

### (4) Production of Immunochromatographic Device

To a polypropylene backing sheet (manufactured by Lohmann Tape Group) as a base material, the conjugate pad produced in (2) above, the antibody-coated membrane produced in (3) above, a glass fiber sample pad, and a cellulose absorption pad were bonded in such a manner that the sample pad, the conjugate pad, the antibody-coated membrane, and the absorption pad were superposed one on another in this order from upstream. Using a cutting machine, the resulting product was cut to a width of 4 mm to produce an immunochromatographic device 4 mm wide and 60 mm long.

### (5) Preparation of Developing Solution

To a 5 mM citrate buffer, Triton X-100 as a nonionic surfactant and CATINAL SPC-20V-S as a cationic surfactant were added at 1.5 wt % and 5 wt % respectively, and sodium chloride was further added at 150 mM to prepare a developing solution. The resulting developing solution contained tri(polyoxyethylene)stearyl ammonium chloride (5 E.O.) at 1 wt %.

### (6) Evaluation of Detecting Capability

To a PBS solution of a SARS-COV-2 nucleocapsid protein (NP) antigen (1 mg/mL), the developing solution was added for dilution in 10-fold steps to obtain a 10 ng/mL positive sample (having an antigen concentration of 10 ng/mL). In addition, the developing solution was further added in such a manner that the antigen was diluted in 5-fold steps so as to be at 2 ng/ml and 0.4 ng/mL. Thus, positive samples (having an antigen concentration of 2 ng/mL and 0.4 ng/mL respectively) were prepared.

In addition, a developing solution having no antigen added thereto was used as a negative sample.

When 15 minutes elapsed after 75 μL of the sample was dropped onto the sample pad, the color development intensity of the test line was measured using an immunochromato reader (C10066, manufactured by Hamamatsu Photonics K.K.).

As the color development intensity, less than 5 mABS was rated −; the range of 5 mABS or more and less than 15 mABS was rated +/−; the range of 15 mABS or more and less than 100 mABS was rated +; the range of 100 mABS or more and less than 300 mABS was rated ++; and 300 mABS or more was rated +++. Less than 5 mABS makes it difficult to rate the test line, and 5 mABS or more and less than 15 mABS allows the test line to be visually recognized, but develops a color so light that the test line can be overlooked. Accordingly, the above-described threshold values were set.

### Example 2

This Example was performed in the same manner as Example 1 except 5 wt % CATINAL SPC-20V-S in (5) in Example 1 was changed to 1.33 wt % LIPOTHOQUAD C/12. Here, the developing solution prepared in Example 2 contained cocoalkylbis(2-hydroxyethyl)methylammonium chloride at 1 wt %.

### Example 3

This Example was performed in the same manner as Example 1 except 5 wt % CATINAL SPC-20V-S in (5) in Example 1 was changed to 1.33 wt % LIPOTHOQUAD O/12. Here, the developing solution prepared in Example 3 contained oleylbis(2-hydroxyethyl)methylammonium chloride at 1 wt %.

### Comparative Example 1

This Comparative Example was performed in the same manner as Example 1 except CATINAL SPC-20V-S in (5) in Example 1 was not used.

### Comparative Example 2

This Comparative Example was performed in the same manner as Example 1 except 5 wt % CATINAL SPC-20V-S in (5) in Example 1 was changed to 1 wt % CTAC (cetyltrimethylammonium chloride).

### Comparative Example 3

This Comparative Example was performed in the same manner as Example 1 except 5 wt % CATINAL SPC-20V-S in (5) in Example 1 was changed to 1 wt % STAC (stearyltrimethylammonium chloride).

Table 1 shows: the surfactants used to prepare the developing solutions in Examples 1 to 3 and Comparative Examples 1 to 3; and the evaluation results of the detecting capability. It should be noted that, in all the Tables including Table 1 below (but excluding Tables 15 and 16), the amount (wt %) of the surfactant means the amount of each surfactant (wt %) in the developing solution, not the amount (wt %) of the surfactant in the sample.

TABLE 1

| | Surfactant | | | | Color Development Intensity [mABS] | | | |
|---|---|---|---|---|---|---|---|---|
| | Nonionic | HLB | | HLB | Antigen Concentration [ng/mL] | | | |
| | Surfactant | (Griffin) | Cationic Surfactant | (Davies) | 0 | 0.4 | 2 | 10 |
| Example 1 | Triton X-100 | 13.4 | tri(polyoxyethylene) | 25.4 | N.D. | 30.3 | 129.0 | 435.2 |
| | (1.5 wt %) | | stearylammonium chloride | | − | + | ++ | +++ |
| | | | (5 E.O.) (1 wt %) | | | | | |
| Example 2 | Triton X-100 | 13.4 | cocoalkylbis(2-hydroxyethyl) | 25.8 | N.D. | 46.5 | 178.4 | 449.6 |
| | (1.5 wt %) | | methylammonium chloride | | − | + | ++ | +++ |
| | | | (1 wt %) | | | | | |
| Example 3 | Triton X-100 | 13.4 | oleylbis(2-hydroxyethyl) | 23.4 | N.D. | 28.4 | 101.2 | 415.1 |
| | (1.5 wt %) | | methylammonium chloride | | − | + | ++ | +++ |
| | | | (1 wt %) | | | | | |
| Comparative | Triton X-100 | 13.4 | — | — | N.D. | N.D. | 14.7 | 73.9 |
| Example 1 | (1.5 wt %) | | | | − | − | +/− | + |
| Comparative | Triton X-100 | 13.4 | cetyltrimethylammonium | | N.D. | 13.1 | 58.9 | 230.7 |
| Example 2 | (1.5 wt %) | | chloride (1 wt %) | | − | +/− | + | ++ |
| Comparative | Triton X-100 | 13.4 | stearyltrimethylammonium | | N.D. | 17.0 | 64.1 | 271.3 |
| Example 3 | (1.5 wt %) | | chloride (1 wt %) | | − | + | + | ++ |

As shown in Table 1, the developing solutions in Examples 1 to 3, compared with Comparative Examples 1 to 3, exhibited a high color development intensity when any positive sample was used. The color development intensity tended to increase, depending on the concentration of the antigen. Even when the antigen concentration was low (the antigen concentration was 0.4 ng/mL), Examples 1 to 3 stably exhibited a color development intensity of 15 mABS or more, enabling the coloration of the test line to be visually recognized. As described above, the chromatographic developing solution including an alkylene oxide-addition cationic surfactant and a nonionic surfactant achieved high-sensitivity chromatography.

### Example 4

This Example was performed in the same manner as Example 1 except 1.5 wt % Triton X-100 in (5) in Example 1 was changed to 1.5 wt % Tween 20.

### Example 5

This Example was performed in the same manner as Example 1 except 1.5 wt % Triton X-100 and 5 wt % CATINAL SPC-20V-S in (5) in Example 1 were changed to 1.5 wt % Tween 20 and 1.33 wt % LIPOTHOQUAD C/12 respectively. Here, the developing solution prepared in Example 5 contained cocoalkylbis(2-hydroxyethyl)methylammonium chloride at 1 wt %.

### Example 6

This Example was performed in the same manner as Example 1 except 1.5 wt % Triton X-100 and 5 wt % CATINAL SPC-20V-S in (5) in Example 1 were changed to 1.5 wt % Tween 20 and 1.33 wt % LIPOTHOQUAD O/12 respectively. Here, the developing solution prepared in Example 6 contained oleylbis(2-hydroxyethyl)methylammonium chloride at 1 wt %.

### Comparative Example 4

This Comparative Example was performed in the same manner as Example 1 except as follows: 1.5 wt % Triton X-100 in (5) in Example 1 was changed to 1.5 wt % Tween 20; and 5 wt % CATINAL SPC-20V-S was not used.

### Comparative Example 5

This Comparative Example was performed in the same manner as Example 1 except 1.5 wt % Triton X-100 and 5 wt % CATINAL SPC-20V-S in (5) in Example 1 were changed to 1.5 wt % Tween 20 and 1 wt % CTAC (cetyltrimethylammonium chloride) respectively.

### Comparative Example 6

This Comparative Example was performed in the same manner as Example 1 except 1.5 wt % Triton X-100 and 5 wt % CATINAL SPC-20V-S in (5) in Example 1 were changed to 1.5 wt % Tween 20 and 1 wt % STAC (stearyltrimethylammonium chloride) respectively.

Table 2 shows: the surfactants used to prepare the developing solutions in Examples 4 to 6 and Comparative Examples 4 to 6; and the evaluation results of the detecting capability.

TABLE 2

| | Surfactant | | | | Color Development Intensity [mABS] | | | |
|---|---|---|---|---|---|---|---|---|
| | Nonionic | HLB | | HLB | Antigen Concentration [ng/mL] | | | |
| | Surfactant | (Griffin) | Cationic Surfactant | (Davies) | 0 | 0.4 | 2 | 10 |
| Example 4 | Tween 20 | 16.7 | tri(polyoxyethylene) | 25.4 | N.D. | 38.4 | 147.3 | 408.7 |
| | (1.5 wt %) | | stearylammonium chloride | | − | + | ++ | +++ |
| | | | (5 E.O.) (1 wt %) | | | | | |
| Example 5 | Tween 20 | 16.7 | cocoalkylbis(2-hydroxyethyl) | 25.8 | N.D. | 53.8 | 185.3 | 474.0 |
| | (1.5 wt %) | | methylammonium chloride | | − | + | ++ | +++ |
| | | | (1 wt %) | | | | | |
| Example 6 | Tween 20 | 16.7 | oleylbis(2-hydroxyethyl) | 23.4 | N.D. | 23.4 | 101.4 | 305.5 |
| | (1.5 wt %) | | methylammonium chloride | | − | + | ++ | +++ |
| | | | (1 wt %) | | | | | |
| Comparative | Tween 20 | 16.7 | — | — | N.D. | N.D. | 16.7 | 89.5 |
| Example 4 | (1.5 wt %) | | | | − | − | +/− | + |
| Comparative | Tween 20 | 16.7 | cetyltrimethylammonium | | 5.3 | 13.5 | 60.1 | 259.7 |
| Example 5 | (1.5 wt %) | | chloride (1 wt %) | | +/− | +/− | + | ++ |
| Comparative | Tween 20 | 16.7 | stearyltrimethylammonium | | 1.0 | 14.5 | 58.2 | 262.5 |
| Example 6 | (1.5 wt %) | | chloride (1 wt %) | | − | +/− | + | ++ |

As shown in Table 2, the developing solutions in Examples 4 to 6, compared with Comparative Examples 4 to 6, exhibited a high color development intensity when any positive sample was used. The color development intensity tended to increase, depending on the concentration of the antigen. Even when the antigen concentration was low (the antigen concentration was 0.4 ng/ml), Examples 4 to 6 stably exhibited a color development intensity of 15 mABS or more, enabling the coloration of the test line to be visually recognized. As described above, the chromatographic developing solution including an alkylene oxide-addition cationic surfactant and a nonionic surfactant achieved high-sensitivity chromatography.

### Example 7

This Example was performed in the same manner as Example 1 except 1.5 wt % Triton X-100 in (5) in Example 1 was changed to 1.5 wt % EMULGEN 150.

### Example 8

This Example was performed in the same manner as Example 1 except 1.5 wt % Triton X-100 and 5 wt % CATINAL SPC-20V-S in (5) in Example 1 were changed to 1.5 wt % EMULGEN 150 and 1.33 wt % LIPOTHOQUAD C/12 respectively. Here, the developing solution prepared in Example 8 contains cocoalkylbis(2-hydroxyethyl)methylammonium chloride at 1 wt %.

### Example 9

This Example was performed in the same manner as Example 1 except 1.5 wt % Triton X-100 and 5 wt % CATINAL SPC-20V-S in (5) in Example 1 were changed to 1.5 wt % EMULGEN 150 and 1.33 wt % LIPOTHOQUAD O/12 respectively. Here, the developing solution prepared in Example 9 contains oleylbis(2-hydroxyethyl)methylammonium chloride at 1 wt %.

### Comparative Example 7

This Comparative Example was performed in the same manner as Example 1 except as follows: 1.5 wt % Triton X-100 in (5) in Example 1 was changed to 1.5 wt % EMULGEN 150; and 5 wt % CATINAL SPC-20V-S was not used.

### Comparative Example 8

This Comparative Example was performed in the same manner as Example 1 except 1.5 wt % Triton X-100 and 5 wt % CATINAL SPC-20V-S in (5) in Example 1 were changed to 1.5 wt % EMULGEN 150 and 1 wt % CTAC (cetyltrimethylammonium chloride) respectively.

### Comparative Example 9

This Comparative Example was performed in the same manner as Example 1 except 1.5 wt % Triton X-100 and 5 wt % CATINAL SPC-20V-S in (5) in Example 1 were changed to 1.5 wt % EMULGEN 150 and 1 wt % STAC (stearyltrimethylammonium chloride) respectively.

Table 3 shows: the surfactants used to prepare the developing solutions in Examples 7 to 9 and Comparative Examples 7 to 9; and the evaluation results of the detecting capability.

TABLE 3

| | Surfactant | | | | Color Development Intensity [mABS] | | | |
|---|---|---|---|---|---|---|---|---|
| | Nonionic | HLB | | HLB | Antigen Concentration [ng/mL] | | | |
| | Surfactant | (Griffin) | Cationic Surfactant | (Davies) | 0 | 0.4 | 2 | 10 |
| Example 7 | EMULGEN 150 | 18.4 | tri(polyoxyethylene) | 25.4 | 3.6 | 50.2 | 181.0 | 445.8 |
| | (1.5 wt %) | | stearylammonium chloride | | − | + | ++ | +++ |
| | | | (5 E.O.) (1 wt %) | | | | | |
| Example 8 | EMULGEN 150 | 18.4 | cocoalkylbis(2-hydroxyethyl) | 25.8 | N.D. | 24.3 | 91.2 | 253.1 |
| | (1.5 wt %) | | methylammonium chloride | | − | + | + | ++ |
| | | | (1 wt %) | | | | | |
| Example 9 | EMULGEN 150 | 18.4 | oleylbis(2-hydroxyethyl) | 23.4 | 3.8 | 32.0 | 118.2 | 377.6 |
| | (1.5 wt %) | | methylammonium chloride | | − | + | ++ | +++ |
| | | | (1 wt %) | | | | | |
| Comparative | EMULGEN 150 | 18.4 | — | — | N.D. | 4.2 | 12.5 | 71.5 |
| Example 7 | (1.5 wt %) | | | | − | − | +/− | + |
| Comparative | EMULGEN 150 | 18.4 | cetyltrimethylammonium | | 6.9 | 7.4 | 26.9 | 186.3 |
| Example 8 | (1.5 wt %) | | chloride (1 wt %) | | +/− | +/− | + | ++ |
| Comparative | EMULGEN 150 | 18.4 | stearyltrimethylammonium | | 3.9 | 4.5 | 31.4 | 188.2 |
| Example 9 | (1.5 wt %) | | chloride (1 wt %) | | − | − | + | ++ |

As shown in Table 3, the developing solutions in Examples 7 to 9, compared with Comparative Examples 7 to 9, exhibited high color development intensity when any positive sample was used. The color development intensity tended to increase, depending on the concentration of the antigen. Even when the antigen concentration was low (the antigen concentration was 0.4 ng/ml), Examples 7 to 9 stably exhibited a color development intensity of 15 mABS or more, enabling the coloration of the test line to be visually recognized. As described above, the chromatographic developing solution including an alkylene oxide-addition cationic surfactant and a nonionic surfactant achieved high-sensitivity chromatography.

### Example 10

This Example was performed in the same manner as Example 1 except as follows: 1.5 wt % Triton X-100 in (5) in Example 1 was changed to 1.5 wt % EMULGEN 108; and only a positive sample having an antigen concentration of 10 ng/mL was prepared as the sample in (6) in Example 1.

### Example 11

This Example was performed in the same manner as Example 1 except as follows: 1.5 wt % Triton X-100 and 5 wt % CATINAL SPC-20V-S in (5) in Example 1 were changed to 1.5 wt % EMULGEN 108 and 1.33 wt % LIPOTHOQUAD C/12 respectively; and only a positive sample having an antigen concentration of 10 ng/mL was prepared as the sample in (6) in Example 1. Here, the developing solution prepared in Example 11 contains cocoalkylbis(2-hydroxyethyl)methylammonium chloride at 1 wt %.

### Comparative Example 10

This Comparative Example was performed in the same manner as Example 1 except as follows: 1.5 wt % Triton X-100 in (5) in Example 1 was changed to 1.5 wt % EMULGEN 108; 5 wt % CATINAL SPC-20V-S was not used; and only a positive sample having an antigen concentration of 10 ng/mL was prepared as the sample in (6) in Example 1.

### Comparative Example 11

This Comparative Example was performed in the same manner as Example 1 except as follows: 1.5 wt % Triton X-100 and 5 wt % CATINAL SPC-20V-S in (5) in Example 1 were changed to 1.5 wt % EMULGEN 108 and 1 wt % CTAC (cetyltrimethylammonium chloride) respectively; and only a positive sample having an antigen concentration of 10 ng/mL was prepared as the sample in (6) in Example 1.

### Comparative Example 12

This Comparative Example was performed in the same manner as Example 1 except as follows: 1.5 wt % Triton X-100 and 5 wt % CATINAL SPC-20V-S in (5) in Example 1 were changed to 1.5 wt % EMULGEN 108 and 1 wt % STAC (stearyltrimethylammonium chloride) respectively; and only a positive sample having an antigen concentration of 10 ng/mL was prepared as the sample in (6) in Example 1.

Table 4 shows: the surfactants used to prepare the developing solutions in Examples 10 and 11 and Comparative Examples 10 to 12; and the evaluation results of the detecting capability.

TABLE 4

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Example 10 | EMULGEN 108 | 12.1 | tri(polyoxyethylene) | 25.4 | 381.6 |
| | (1.5 wt %) | | stearylammonium chloride | | |
| | | | (5 E.O.) (1 wt %) | | |
| Example 11 | EMULGEN 108 | 12.1 | cocoalkylbis(2-hydroxyethyl) | 25.8 | 332.3 |
| | (1.5 wt %) | | methylammonium chloride | | |
| | | 12.1 | (1 wt %) | | |
| Comparative | EMULGEN 108 | | — | — | 29.1 |
| Example 10 | (1.5 wt %) | | | | |
| Comparative | EMULGEN 108 | 12.1 | cetyltrimethylammonium | | 260.6 |
| Example 11 | (1.5 wt %) | | chloride (1 wt %) | | |
| Comparative | EMULGEN 108 | 12.1 | stearyltrimethylammonium | | 48.6 |
| Example 12 | (1.5 wt %) | | chloride (1 wt %) | | |

As shown in Table 4, the developing solutions in Examples 10 and 11, compared with Comparative Examples 10 to 12, exhibited a high color development intensity. As described above, the chromatographic developing solution including an alkylene oxide-addition cationic surfactant and a nonionic surfactant achieved high-sensitivity chromatography.

### Example 12

This Example was performed in the same manner as Example 1 except as follows: 1.5 wt % Triton X-100 in (5) in Example 1 was changed to 1.5 wt % Brij 35; and only a positive sample having an antigen concentration of 10 ng/mL was prepared as the sample in (6) in Example 1.

### Example 13

This Example was performed in the same manner as Example 1 except as follows: 1.5 wt % Triton X-100 and 5 wt % CATINAL SPC-20V-S in (5) in Example 1 were changed to 1.5 wt % Brij 35 and 1.33 wt % LIPOTHOQUAD C/12 respectively; and only a positive sample having an antigen concentration of 10 ng/mL was prepared as the sample in (6) in Example 1. Here, the developing solution prepared in Example 13 contains cocoalkylbis(2-hydroxyethyl)methylammonium chloride at 1 wt %.

### Comparative Example 13

This Comparative Example was performed in the same manner as Example 1 except as follows: 1.5 wt % Triton X-100 in (5) in Example 1 was changed to 1.5 wt % Brij 35; 5 wt % CATINAL SPC-20V-S was not used; and only a positive sample having an antigen concentration of 10 ng/mL was prepared as the sample in (6) in Example 1.

### Comparative Example 14

This Comparative Example was performed in the same manner as Example 1 except as follows: 1.5 wt % Triton X-100 and 5 wt % CATINAL SPC-20V-S in (5) in Example 1 were changed to 1.5 wt % Brij 35 and 1 wt % CTAC (cetyltrimethylammonium chloride) respectively; and only a positive sample having an antigen concentration of 10 ng/mL was prepared as the sample in (6) in Example 1.

### Comparative Example 15

This Comparative Example was performed in the same manner as Example 1 except as follows: 1.5 wt % Triton X-100 and 5 wt % CATINAL SPC-20V-S in (5) in Example 1 were changed to 1.5 wt % Brij 35 and 1 wt % STAC (stearyltrimethylammonium chloride) respectively; and only a positive sample having an antigen concentration of 10 ng/ml was prepared as the sample in (6) in Example 1.

Table 5 shows: the surfactants used to prepare the developing solutions in Examples 12 and 13 and Comparative Examples 13 to 15; and the evaluation results of the detecting capability.

TABLE 5

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Example 12 | Brij 35 | 16.9 | tri(polyoxyethylene) | 25.4 | 442.6 |
| | (1.5 wt %) | | stearylammonium chloride | | |
| | | | (5 E.O.) (1 wt %) | | |
| Example 13 | Brij 35 | 16.9 | cocoalkylbis(2-hydroxyethyl) | 25.8 | 414.0 |
| | (1.5 wt %) | | methylammonium chloride | | |
| | | | (1 wt %) | | |
| Comparative | Brij 35 | 16.9 | — | — | 67.2 |
| Example 13 | (1.5 wt %) | | | | |
| Comparative | Brij 35 | 16.9 | cetyltrimethylammonium | | 348.5 |
| Example 14 | (1.5 wt %) | | chloride (1 wt %) | | |
| Comparative | Brij 35 | 16.9 | stearyltrimethylammonium | | 345.5 |
| Example 15 | (1.5 wt %) | | chloride (1 wt %) | | |

As shown in Table 5, the developing solutions in Examples 12 and 13, compared with Comparative Examples 13 to 15, exhibited a high color development intensity. As described above, the chromatographic developing solution including an alkylene oxide-addition cationic surfactant and a nonionic surfactant achieved high-sensitivity chromatography.

### Example 14

This Example was performed in the same manner as Example 1 except as follows: 1.5 wt % Triton X-100 in (5) in Example 1 was changed to 1.5 wt % AMIET 105A; and only a positive sample having an antigen concentration of 10 ng/mL was prepared as the sample in (6) in Example 1.

### Example 15

This Example was performed in the same manner as Example 1 except as follows: 1.5 wt % Triton X-100 and 5 wt % CATINAL SPC-20V-S in (5) in Example 1 were changed to 1.5 wt % AMIET 105A and 1.33 wt % LIPOTHOQUAD O/12 respectively; and only a positive sample having an antigen concentration of 10 ng/mL was prepared as the sample in (6) in Example 1. Here, the developing solution prepared in Example 15 contained oleylbis(2-hydroxyethyl)methylammonium chloride at 1 wt %.

### Comparative Example 16

This Comparative Example was performed in the same manner as Example 1 except as follows: 1.5 wt % Triton X-100 in (5) in Example 1 was changed to 1.5 wt % AMIET 105A; 5 wt % CATINAL SPC-20V-S was not used; and only a positive sample having an antigen concentration of 10 ng/mL was prepared as the sample in (6) in Example 1.

### Comparative Example 17

This Comparative Example was performed in the same manner as Example 1 except as follows: 1.5 wt % Triton X-100 and 5 wt % CATINAL SPC-20V-S in (5) in Example 1 were changed to 1.5 wt % AMIET 105A and 1 wt % CTAC (cetyltrimethylammonium chloride) respectively; and only a positive sample having an antigen concentration of 10 ng/ml was prepared as the sample in (6) in Example 1.

### Comparative Example 18

This Comparative Example was performed in the same manner as Example 1 except as follows: 1.5 wt % Triton X-100 and 5 wt % CATINAL SPC-20V-S in (5) in Example 1 were changed to 1.5 wt % AMIET 105A and 1 wt % STAC (stearyltrimethylammonium chloride) respectively; and only a positive sample having an antigen concentration of 10 ng/ml was prepared as the sample in (6) in Example 1.

Table 6 shows: the surfactants used to prepare the developing solutions in Examples 14 and 15 and Comparative Examples 16 to 18; and the evaluation results of the detecting capability.

TABLE 6

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Example 14 | AMIET105A | 10.8 | tri(polyoxyethylene) | 25.4 | 172.8 |
| | (1.5 wt %) | | stearylammonium chloride | | |
| | | | (5 E.O.) (1 wt %) | | |
| Example 15 | AMIET105A | 10.8 | oleylbis(2-hydroxyethyl) | 23.4 | 429.8 |
| | (1.5 wt %) | | methylammonium chloride | | |
| | | | (1 wt %) | | |
| Comparative | AMIET105A | 10.8 | — | — | 28.2 |
| Example 16 | (1.5 wt %) | | | | |
| Comparative | AMIET105A | 10.8 | cetyltrimethylammonium | | 6.4 |
| Example 17 | (1.5 wt %) | | chloride (1 wt %) | | |
| Comparative | AMIET105A | 10.8 | stearyltrimethylammonium | | 6.3 |
| Example 18 | (1.5 wt %) | | chloride (1 wt %) | | |

As shown in Table 6, the developing solutions in Examples 14 and 15, compared with Comparative Examples 16 to 18, exhibited a high color development intensity. As described above, the chromatographic developing solution including an alkylene oxide-addition cationic surfactant and a nonionic surfactant achieved high-sensitivity chromatography.

### Comparative Example 19

This Comparative Example was performed in the same manner as Example 1 except as follows: 5 wt % CATINAL SPC-20V-S in (5) in Example 1 was changed to 1 wt % AMIET 105A; and only a positive sample having an antigen concentration of 10 ng/ml was prepared as the sample in (6) in Example 1.

Table 7 shows: the surfactants used to prepare the developing solutions in Comparative Example 19; and the evaluation results of the detecting capability. Table 7 additionally shows a weighted-average HLB value calculated from the HLB value and amount of Triton X-100 and the HLB value and amount of AMIET 105A.

TABLE 7

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Comparative | Triton X-100 | 13.4 | — | — | 47.4 |
| Example 19 | (1.5 wt %) | | | | |
| | AMIET105A | 10.8 | | | |
| | (1 wt %) | | | | |
| | weighted-average | 12.4 | | | |
| | HLB | | | | |

In Comparative Example 19, the alkylene oxide-addition cationic surfactant was not used, and instead, a nonionic surfactant having a polyoxyethylene structure (alkylene oxide structure) was used. Comparative Example 19 suggests that using the alkylene oxide-addition cationic surfactant has superiority over simply using an alkylene oxide-addition surfactant.

### Example 16

This Example was performed in the same manner as Example 1 except as follows: only a positive sample having an antigen concentration of 10 ng/mL was prepared as the positive sample in (6) in Example 1; a developing solution having no antigen added thereto was used as a negative sample; and 75 μL of the sample was dropped onto a sample pad, after which the color development intensity of the test line was measured every 30 seconds using an immunochromato reader until 15 minutes elapsed.

### Example 17

This Example was performed in the same manner as Example 1 except as follows: 5 wt % CATINAL SPC-20V-S in (5) in Example 1 was changed to 1.33 wt % LIPOTHOQUAD C/12; only a positive sample having an antigen concentration of 10 ng/mL was prepared as the positive sample in (6) in Example 1; a developing solution having no antigen added thereto was used as a negative sample; and 75 μL of the sample was dropped onto a sample pad, after which the color development intensity of the test line was measured every 30 seconds using an immunochromato reader until 15 minutes elapsed. Here, the developing solution prepared in Example 17 contained cocoalkylbis(2-hydroxyethyl)methylammonium chloride at 1 wt %.

### Example 18

This Example was performed in the same manner as Example 1 except as follows: 5 wt % CATINAL SPC-20V-S in (5) in Example 1 was changed to 1.33 wt % LIPOTHOQUAD O/12; only a positive sample having an antigen concentration of 10 ng/mL was prepared as the positive sample in (6) in Example 1; a developing solution having no antigen added thereto was used as a negative sample; and 75 μL of the sample was dropped onto a sample pad, after which the color development intensity of the test line was measured every 30 seconds using an immunochromato reader until 15 minutes elapsed. Here, the developing solution prepared in Example 18 contained oleylbis(2-hydroxyethyl)methylammonium chloride at 1 wt %.

### Comparative Example 20

This Comparative Example was performed in the same manner as Example 1 except as follows: 5 wt % CATINAL SPC-20V-S in (5) in Example 1 was not used; only a positive sample having an antigen concentration of 10 ng/mL was prepared as the positive sample in (6) in Example 1; a developing solution having no antigen added thereto was used as a negative sample; and 75 μL of the sample was dropped onto a sample pad, after which the color development intensity of the test line was measured every 30 seconds using an immunochromato reader until 15 minutes elapsed.

### Comparative Example 21

This Comparative Example was performed in the same manner as Example 1 except as follows: 5 wt % CATINAL SPC-20V-S in (5) in Example 1 was changed to 1 wt % CTAC (cetyltrimethylammonium chloride); only a positive sample having an antigen concentration of 10 ng/mL was prepared as the positive sample in (6) in Example 1; a developing solution having no antigen added thereto was used as a negative sample; and 75 μL of the sample was dropped onto a sample pad, after which the color development intensity of the test line was measured every 30 seconds using an immunochromato reader until 15 minutes elapsed.

### Comparative Example 22

This Comparative Example was performed in the same manner as Example 1 except as follows: 5 wt % CATINAL SPC-20V-S in (5) in Example 1 was changed to 1 wt % STAC (stearyltrimethylammonium chloride); only a positive sample having an antigen concentration of 10 ng/mL was prepared as the positive sample in (6) in Example 1; a developing solution having no antigen added thereto was used as a negative sample; and 75 μL of the sample was dropped onto a sample pad, after which the color development intensity of the test line was measured every 30 seconds using an immunochromato reader until 15 minutes elapsed.

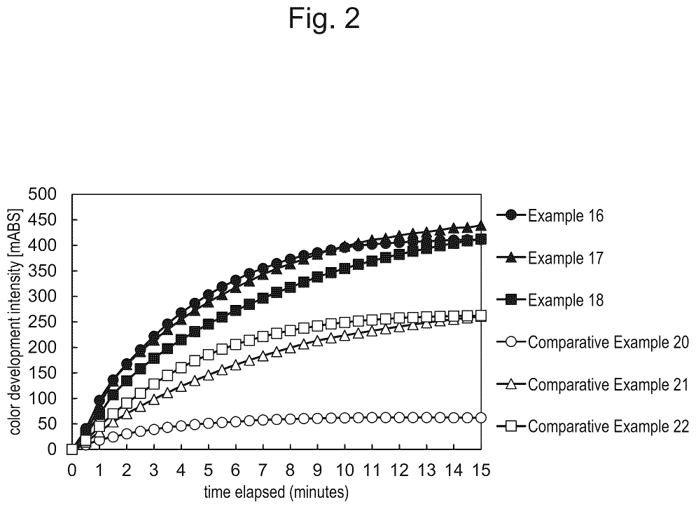

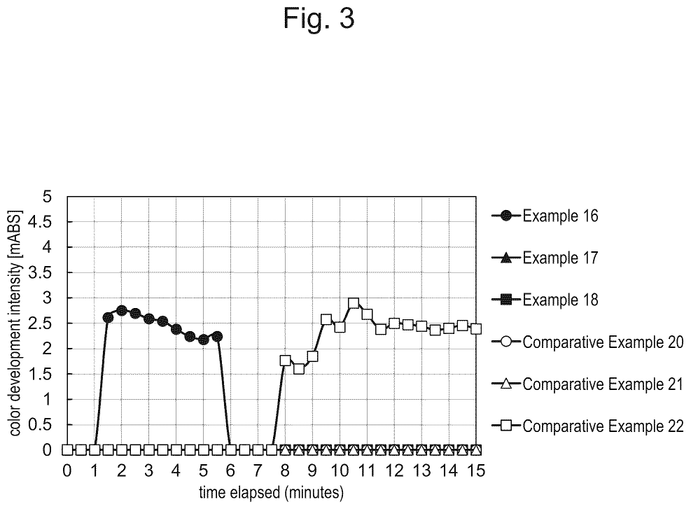

Table 8 shows the surfactants used to prepare the developing solutions in Examples 16 to 18 and Comparative Examples 20 to 22. In addition, FIG. 2 illustrates the relationship between the color development intensity of a positive sample and the time elapsed, wherein the sample was produced in each of Examples 16 to 18 and Comparative Examples 20 to 22. FIG. 3 illustrates the relationship between the color development intensity of a negative sample and the time elapsed, wherein the sample was produced in each of Examples 16 to 18 and Comparative Examples 20 to 22.

TABLE 8

| | Surfactant | | | |
|---|---|---|---|---|
| | Nonionic | HLB | Cationic | HLB |
| | Surfactant | (Griffin) | Surfactant | (Davies) |
| Example 16 | Triton X-100 | 13.4 | tri(polyoxyethylene) | 25.4 |
| | (1.5 wt %) | | stearylammonium chloride | |
| | | | (5 E.O.) (1 wt %) | |
| Example 17 | Triton X-100 | 13.4 | cocoalkylbis(2-hydroxyethyl) | 25.8 |
| | (1.5 wt %) | | methylammonium chloride | |
| | | | (1 wt %) | |
| Example 18 | Triton X-100 | 13.4 | oleylbis(2-hydroxyethyl) | 23.4 |
| | (1.5 wt %) | | methylammonium chloride | |
| | | | (1 wt %) | |
| Comparative | Triton X-100 | 13.4 | — | — |
| Example 20 | (1.5 wt %) | | | |
| Comparative | Triton X-100 | 13.4 | cetyltrimethylammonium | |
| Example 21 | (1.5 wt %) | | chloride (1 wt %) | |
| Comparative | Triton X-100 | 13.4 | stearyltrimethylammonium | |
| Example 22 | (1.5 wt %) | | chloride (1 wt %) | |

FIG. 2 has revealed that, in Examples 16 to 18, the color development intensity was rapidly increased after the positive sample was dropped onto the sample pad, compared with Comparative Examples 20 to 22. When 30 seconds elapsed after the positive sample was dropped, Examples 16 to 18 exhibited a color development intensity of 15 mABS or more, enabling the coloration of the test line to be visually recognized. In addition, the color development intensities in Examples 16 to 18 were larger than in Comparative Examples 20 to 22 in the same time elapsed. According to FIG. 3, Examples 16 to 18 and Comparative Examples 20 to 22 exhibited a color development intensity of less than 5 mABS after the negative sample was dropped onto the sample pad. The coloration of the test line was not observed. As described above, the chromatographic developing solution including an alkylene oxide-addition cationic surfactant and a nonionic surfactant achieved high-sensitivity chromatography.

### Comparative Example 23

This Comparative Example was performed in the same manner as Example 1 except as follows: 1.5 wt % Triton X-100 in (5) in Example 1 was not used; only a positive sample having an antigen concentration of 10 ng/mL was prepared as the positive sample in (6) in Example 1; and a developing solution having no antigen added thereto was used as a negative sample.

### Example 19

This Example was performed in the same manner as Example 1 except as follows: 1.5 wt % Triton X-100 in (5) in Example 1 was changed to 0.75 wt % Triton X-100; only a positive sample having an antigen concentration of 10 ng/mL was prepared as the positive sample in (6) in Example 1; and a developing solution having no antigen added thereto was used as a negative sample.

### Example 20

This Example was performed in the same manner as Example 1 except as follows: 1.5 wt % Triton X-100 in (5) in Example 1 was changed to 1 wt % Triton X-100; only a positive sample having an antigen concentration of 10 ng/mL was prepared as the positive sample in (6) in Example 1; and a developing solution having no antigen added thereto was used as a negative sample.

### Example 21

This Example was performed in the same manner as Example 1 except as follows: only a positive sample having an antigen concentration of 10 ng/mL was prepared as the positive sample in (6) in Example 1; and a developing solution having no antigen added thereto was used as a negative sample.

### Example 22

This Example was performed in the same manner as Example 1 except as follows: 1.5 wt % Triton X-100 in (5) in Example 1 was changed to 2 wt % Triton X-100; only a positive sample having an antigen concentration of 10 ng/mL was prepared as the positive sample in (6) in Example 1; and a developing solution having no antigen added thereto was used as a negative sample.

### Example 23

This Example was performed in the same manner as Example 1 except as follows: 1.5 wt % Triton X-100 in (5) in Example 1 was changed to 3 wt % Triton X-100; only a positive sample having an antigen concentration of 10 ng/mL was prepared as the positive sample in (6) in Example 1; and a developing solution having no antigen added thereto was used as a negative sample.

### Example 24

This Example was performed in the same manner as Example 1 except as follows: 1.5 wt % Triton X-100 in (5) in Example 1 was changed to 5 wt % Triton X-100; only a positive sample having an antigen concentration of 10 ng/mL was prepared as the positive sample in (6) in Example 1; and a developing solution having no antigen added thereto was used as a negative sample.

### Example 25

This Example was performed in the same manner as Example 1 except as follows: 1.5 wt % Triton X-100 in (5) in Example 1 was changed to 7.5 wt % Triton X-100; only a positive sample having an antigen concentration of 10 ng/mL was prepared as the positive sample in (6) in Example 1; and a developing solution having no antigen added thereto was used as a negative sample.

### Example 26

This Example was performed in the same manner as Example 1 except as follows: 1.5 wt % Triton X-100 in (5) in Example 1 was changed to 10 wt % Triton X-100; only a positive sample having an antigen concentration of 10 ng/mL was prepared as the positive sample in (6) in Example 1; and a developing solution having no antigen added thereto was used as a negative sample.

Table 9 shows: the surfactants used to prepare developing solutions in Examples 19 to 26 and Comparative Example 23; the ratio (weight ratio) of concentration between the nonionic surfactant and the cationic surfactant (the nonionic surfactant concentration/the cationic surfactant concentration); and the evaluation results of the detecting capability.

TABLE 9

| | | | | | Nonionic | | |
|---|---|---|---|---|---|---|---|
| | | | | | Surfactant/ | Color Development | |
| | | | | | Cationic | Intensity [mABS] | |
| | Surfactant | | | | Surfactant | Antigen | |
| | Nonionic | HLB | | HLB | Ratio by | Concentration [ng/mL] | |
| | Surfactant | (Griffin) | Cationic Surfactant | (Davies) | Concentration | 0 | 10 |
| Comparative | — | | tri(polyoxyethylene) | 25.4 | — | N.D. | 10.0 |
| Example 23 | | | stearylammonium chloride | | | Invalid | Invalid |
| | | | (5 E.O.) (1 wt %) | | | | |
| Example 19 | Triton X-100 | 13.4 | tri(polyoxyethylene) | 25.4 | 0.75 | 4.0 | 371.8 |
| | (0.75 wt %) | | stearylammonium chloride | | | | |
| | | | (5 E.O.) (1 wt %) | | | | |
| Example 20 | Triton X-100 | 13.4 | tri(polyoxyethylene) | 25.4 | 1 | 1.8 | 491.9 |
| | (1 wt %) | | stearylammonium chloride | | | | |
| | | | (5 E.O.) (1 wt %) | | | | |
| Example 21 | Triton X-100 | 13.4 | tri(polyoxyethylene) | 25.4 | 1.5 | 1.3 | 516.1 |
| | (1.5 wt %) | | stearylammonium chloride | | | | |
| | | | (5 E.O.) (1 wt %) | | | | |
| Example 22 | Triton X-100 | 13.4 | tri(polyoxyethylene) | 25.4 | 2 | N.D. | 434.4 |
| | (2 wt %) | | stearylammonium chloride | | | | |
| | | | (5 E.O.) (1 wt %) | | | | |
| Example 23 | Triton X-100 | 13.4 | tri(polyoxyethylene) | 25.4 | 3 | N.D. | 312.5 |
| | (3 wt %) | | stearylammonium chloride | | | | |
| | | | (5 E.O.) (1 wt %) | | | | |
| Example 24 | Triton X-100 | 13.4 | tri(polyoxyethylene) | 25.4 | 5 | N.D. | 133.5 |
| | (5 wt %) | | stearylammonium chloride | | | | |
| | | | (5 E.O.) (1 wt %) | | | | |
| Example 25 | Triton X-100 | 13.4 | tri(polyoxyethylene) | 25.4 | 7.5 | N.D. | 75.5 |
| | (7.5 wt %) | | stearylammonium chloride | | | | |
| | | | (5 E.O.) (1 wt %) | | | | |
| Example 26 | Triton X-100 | 13.4 | tri(polyoxyethylene) | 25.4 | 10 | N.D. | 77.0 |
| | (10 wt %) | | stearylammonium chloride | | | | |
| | | | (5 E.O.) (1 wt %) | | | | |

As shown in Table 9, the developing solutions in Examples 19 to 26, compared with Comparative Example 23, exhibited a high color development intensity when any positive sample was used. As described above, the chromatographic developing solution including an alkylene oxide-addition cationic surfactant and a nonionic surfactant achieved high-sensitivity chromatography.

In Comparative Example 23, coloration derived from the labeling substance or the composite of an analyte and a labeling substance was observed at or around the boundary between the labeling substance-retaining section and the solid-phase support, no coloration was observed on the control line, and no coloration in line form was observed on the test line (not shown). This is considered to be because the alkylene oxide-addition cationic surfactant excessively aggregated the labeling substance or the composite of an analyte and a labeling substance, and thus, the aggregate formed was not allowed to be moved in the solid-phase support by the capillarity phenomena, resulting in staying at or around the boundary between the labeling substance-retaining section and the solid-phase support. On the other hand, in Examples 19 to 26, no coloration derived from the labeling substance or the composite of an analyte and a labeling substance was observed at or around the boundary between the labeling substance-retaining section and the solid-phase support (not shown). This is considered to be because the alkylene oxide-addition cationic surfactant promoted the aggregation of the labeling substance or the composite of an analyte and a labeling substance, and besides, the nonionic surfactant inhibited excessive aggregation of the labeling substance or the composite of an analyte and a labeling substance, so that the aggregate formed moved in the solid-phase support owing to the capillarity phenomena, and trapped by the trapping substance in the test line. The above-described results suggest that using the alkylene oxide-addition cationic surfactant and the nonionic surfactant together has superiority over using the alkylene oxide-addition surfactant singly.

### Comparative Example 24

This Comparative Example was performed in the same manner as Example 1 except as follows: 1.5 wt % Triton X-100 in (5) in Example 1 was changed to 2 wt % Triton X-100; 5 wt % CATINAL SPC-20V-S was not used; only a positive sample having an antigen concentration of 10 ng/mL was prepared as the positive sample in (6) in Example 1; and a developing solution having no antigen added thereto was used as a negative sample.

### Example 27

This Example was performed in the same manner as Example 1 except as follows: 1.5 wt % Triton X-100 and 5 wt % CATINAL SPC-20V-S in (5) in Example 1 were changed to 2 wt % Triton X-100 and 0.5 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 27 contained tri(polyoxyethylene)stearyl ammonium chloride (5 E.O.) at 0.1 wt %.

### Example 28

This Example was performed in the same manner as Example 1 except as follows: 1.5 wt % Triton X-100 and 5 wt % CATINAL SPC-20V-S in (5) in Example 1 were changed to 2 wt % Triton X-100 and 1 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 28 contained tri(polyoxyethylene)stearyl ammonium chloride (5 E.O.) at 0.2 wt %.

### Example 29

This Example was performed in the same manner as Example 1 except as follows: 1.5 wt % Triton X-100 and 5 wt % CATINAL SPC-20V-S in (5) in Example 1 were changed to 2 wt % Triton X-100 and 2.5 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 29 contained tri(polyoxyethylene)stearyl ammonium chloride (5 E.O.) at 0.5 wt %.

### Example 30

This Example was performed in the same manner as Example 1 except as follows: 1.5 wt % Triton X-100 in (5) in Example 1 was changed to 2 wt % Triton X-100; only a positive sample having an antigen concentration of 10 ng/mL was prepared as the positive sample in (6) in Example 1; and a developing solution having no antigen added thereto was used as a negative sample.

### Example 31

This Example was performed in the same manner as Example 1 except as follows: 1.5 wt % Triton X-100 and 5 wt % CATINAL SPC-20V-S in (5) in Example 1 were changed to 2 wt % Triton X-100 and 7.5 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 31 contained tri(polyoxyethylene)stearyl ammonium chloride (5 E.O.) at 1.5 wt %.

### Example 32

This Example was performed in the same manner as Example 1 except as follows: 1.5 wt % Triton X-100 and 5 wt % CATINAL SPC-20V-S in (5) in Example 1 were changed to 2 wt % Triton X-100 and 10 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 32 contained tri(polyoxyethylene)stearyl ammonium chloride (5 E.O.) at 2 wt %.

### Example 33

This Example was performed in the same manner as Example 1 except as follows: 1.5 wt % Triton X-100 and 5 wt % CATINAL SPC-20V-S in (5) in Example 1 were changed to 2 wt % Triton X-100 and 15 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 33 contained tri(polyoxyethylene)stearyl ammonium chloride (5 E.O.) at 3 wt %.

### Example 34

This Example was performed in the same manner as Example 1 except as follows: 1.5 wt % Triton X-100 and 5 wt % CATINAL SPC-20V-S in (5) in Example 1 were changed to 2 wt % Triton X-100 and 30 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 34 contained tri(polyoxyethylene)stearyl ammonium chloride (5 E.O.) at 6 wt %.

### Example 35

This Example was performed in the same manner as Example 1 except as follows: 1.5 wt % Triton X-100 and 5 wt % CATINAL SPC-20V-S in (5) in Example 1 were changed to 2 wt % Triton X-100 and 50 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 35 contained tri(polyoxyethylene)stearyl ammonium chloride (5 E.O.) at 10 wt %.

### Example 36

This Example was performed in the same manner as Example 1 except as follows: 1.5 wt % Triton X-100 and 5 wt % CATINAL SPC-20V-S in (5) in Example 1 were changed to 2 wt % Triton X-100 and 62.5 wt % CATINAL SPC-20V-S respectively; only a positive sample having an antigen concentration of 10 ng/mL was prepared as the positive sample in (6) in Example 1; and a developing solution having no antigen added thereto was used as a negative sample. Here, the developing solution prepared in Example 36 contained tri(polyoxyethylene)stearyl ammonium chloride (5 E.O.) at 12.5 wt %.

Table 10 shows: the surfactants used to prepare developing solutions in Examples 27 to 36 and Comparative Example 24; the ratio (weight ratio) of concentration between the nonionic surfactant and the cationic surfactant (the nonionic surfactant concentration/the cationic surfactant concentration); and the evaluation results of the detecting capability.

TABLE 10

| | | | | | Nonionic | | |
|---|---|---|---|---|---|---|---|
| | | | | | Surfactant/ | Color Development | |
| | | | | | Cationic | Intensity [mABS] | |
| | Surfactant | | | | Surfactant | Antigen | |
| | Nonionic | HLB | | HLB | Ratio by | Concentration [ng/mL] | |
| | Surfactant | (Griffin) | Cationic Surfactant | (Davies) | Concentration | 0 | 10 |
| Comparative | Triton X-100 | 13.4 | — | | — | 1.7 | 163.0 |
| Example 24 | (2 wt %) | | | | | | |
| Example 27 | Triton X-100 | 13.4 | tri(polyoxyethylene) | 25.4 | 20 | N.D. | 185.6 |
| | (2 wt %) | | stearylammonium chloride | | | | |
| | | | (5 E.O.) (0.1 wt %) | | | | |
| Example 28 | Triton X-100 | 13.4 | tri(polyoxyethylene) | 25.4 | 10 | N.D. | 210.5 |
| | (2 wt %) | | stearylammonium chloride | | | | |
| | | | (5 E.O.) (0.2 wt %) | | | | |
| Example 29 | Triton X-100 | 13.4 | tri(polyoxyethylene) | 25.4 | 4 | 0.7 | 272.6 |
| | (2 wt %) | | stearylammonium chloride | | | | |
| | | | (5 E.O.) (0.5 wt %) | | | | |
| Example 30 | Triton X-100 | 13.4 | tri(polyoxyethylene) | 25.4 | 2 | N.D. | 470.3 |
| | (2 wt %) | | stearylammonium chloride | | | | |
| | | | (5 E.O.) (1 wt %) | | | | |
| Example 31 | Triton X-100 | 13.4 | tri(polyoxyethylene) | 25.4 | 1.33 | N.D. | 530.7 |
| | (2 wt %) | | stearylammonium chloride | | | | |
| | | | (5 E.O.) (1.5 wt %) | | | | |
| Example 32 | Triton X-100 | 13.4 | tri(polyoxyethylene) | 25.4 | 1 | N.D. | 559.9 |
| | (2 wt %) | | stearylammonium chloride | | | | |
| | | | (5 E.O.) (2 wt %) | | | | |
| Example 33 | Triton X-100 | 13.4 | tri(polyoxyethylene) | 25.4 | 0.67 | 2.0 | 501.9 |
| | (2 wt %) | | stearylammonium chloride | | | | |
| | | | (5 E.O.) (3 wt %) | | | | |
| Example 34 | Triton X-100 | 13.4 | tri(polyoxyethylene) | 25.4 | 0.33 | N.D. | 396.4 |
| | (2 wt %) | | stearylammonium chloride | | | | |
| | | | (5 E.O.) (6 wt %) | | | | |
| Example 35 | Triton X-100 | 13.4 | tri(polyoxyethylene) | 25.4 | 0.2 | N.D. | 262.2 |
| | (2 wt %) | | stearylammonium chloride | | | | |
| | | | (5 E.O.) (10 wt %) | | | | |
| Example 36 | Triton X-100 | 13.4 | tri(polyoxyethylene) | 25.4 | 0.16 | N.D. | 196.0 |
| | (2 wt %) | | stearylammonium chloride | | | | |
| | | | (5 E.O.) (12.5 wt %) | | | | |

As shown in Table 10, the developing solutions in Examples 27 to 36, compared with Comparative Example 24, exhibited a high color development intensity when any positive sample was used. As described above, the chromatographic developing solution including an alkylene oxide-addition cationic surfactant and a nonionic surfactant achieved high-sensitivity chromatography.

### Example 37

This Example was performed in the same manner as Example 1 except as follows: 1.5 wt % Triton X-100 and 5 wt % CATINAL SPC-20V-S in (5) in Example 1 were changed to 2 wt % Triton X-100 and 1.33 wt % LIPOTHOQUAD C/12 respectively; and only a positive sample having an antigen concentration of 10 ng/mL was prepared as the sample in (6) in Example 1. Here, the developing solution prepared in Example 37 contained cocoalkylbis(2-hydroxyethyl)methylammonium chloride at 1 wt %.

### Example 38

This Example was performed in the same manner as Example 1 except as follows: 1.5 wt % Triton X-100 and 5 wt % CATINAL SPC-20V-S in (5) in Example 1 were changed to 2 wt % Triton X-100 and 2 wt % LIPOTHOQUAD C/12 respectively; and only a positive sample having an antigen concentration of 10 ng/mL was prepared as the sample in (6) in Example 1. Here, the developing solution prepared in Example 38 contained cocoalkylbis(2-hydroxyethyl)methylammonium chloride at 1.5 wt %.

### Example 39

This Example was performed in the same manner as Example 1 except as follows: 1.5 wt % Triton X-100 and 5 wt % CATINAL SPC-20V-S in (5) in Example 1 were changed to 2 wt % Triton X-100 plus 1 wt % Tween 20 and 2 wt % LIPOTHOQUAD C/12 respectively; and only a positive sample having an antigen concentration of 10 ng/mL was prepared as the sample in (6) in Example 1. Here, the developing solution prepared in Example 39 contained cocoalkylbis(2-hydroxyethyl)methylammonium chloride at 1.5 wt %.

### Example 40

This Example was performed in the same manner as Example 1 except as follows: 1.5 wt % Triton X-100 and 5 wt % CATINAL SPC-20V-S in (5) in Example 1 were changed to 2 wt % Triton X-100 plus 1 wt % EMULGEN 150 and 2 wt % LIPOTHOQUAD C/12 respectively; and only a positive sample having an antigen concentration of 10 ng/ml was prepared as the sample in (6) in Example 1. Here, the developing solution prepared in Example 40 contained cocoalkylbis(2-hydroxyethyl)methylammonium chloride at 1.5 wt %.

### Example 41

This Example was performed in the same manner as Example 1 except as follows: 1.5 wt % Triton X-100 and 5 wt % CATINAL SPC-20V-S in (5) in Example 1 were changed to 2 wt % Triton X-100 plus 1 wt % EMULGEN 108 and 2 wt % LIPOTHOQUAD C/12 respectively; and only a positive sample having an antigen concentration of 10 ng/mL was prepared as the sample in (6) in Example 1. Here, the developing solution prepared in Example 41 contained cocoalkylbis(2-hydroxyethyl)methylammonium chloride at 1.5 wt %.

### Example 42

This Example was performed in the same manner as Example 1 except as follows: 1.5 wt % Triton X-100 and 5 wt % CATINAL SPC-20V-S in (5) in Example 1 were changed to 2 wt % Triton X-100 plus 1 wt % Brij 35 and 2 wt % LIPOTHOQUAD C/12 respectively; and only a positive sample having an antigen concentration of 10 ng/mL was prepared as the sample in (6) in Example 1. Here, the developing solution prepared in Example 42 contained cocoalkylbis(2-hydroxyethyl)methylammonium chloride at 1.5 wt %.

### Example 43

This Example was performed in the same manner as Example 1 except as follows: 1.5 wt % Triton X-100 and 5 wt % CATINAL SPC-20V-S in (5) in Example 1 were changed to 2 wt % Triton X-100 plus 1 wt % AMIET 105A and 2 wt % LIPOTHOQUAD C/12 respectively; and only a positive sample having an antigen concentration of 10 ng/mL was prepared as the sample in (6) in Example 1. Here, the developing solution prepared in Example 43 contained cocoalkylbis(2-hydroxyethyl)methylammonium chloride at 1.5 wt %.

### Example 44

This Example was performed in the same manner as Example 1 except as follows: 1.5 wt % Triton X-100 and 5 wt % CATINAL SPC-20V-S in (5) in Example 1 were changed to 2 wt % Triton X-100 plus 1 wt % AMIET 320 and 2 wt % LIPOTHOQUAD C/12 respectively; and only a positive sample having an antigen concentration of 10 ng/mL was prepared as the sample in (6) in Example 1. Here, the developing solution prepared in Example 44 contained cocoalkylbis(2-hydroxyethyl)methylammonium chloride at 1.5 wt %.

### Example 45

This Example was performed in the same manner as Example 1 except as follows: 1.5 wt % Triton X-100 and 5 wt % CATINAL SPC-20V-S in (5) in Example 1 were changed to 2 wt % Triton X-100 and 5 wt % CATINAL SPC-20V-S plus 1.33 wt % LIPOTHOQUAD C/12 respectively; and only a positive sample having an antigen concentration of 10 ng/mL was prepared as the sample in (6) in Example 1. Here, the developing solution prepared in Example 45 contained tri(polyoxyethylene)stearyl ammonium chloride (5 E.O.) at 1 wt % and cocoalkylbis(2-hydroxyethyl)methylammonium chloride at 1 wt %.

### Example 46

This Example was performed in the same manner as Example 1 except as follows: 1.5 wt % Triton X-100 and 5 wt % CATINAL SPC-20V-S in (5) in Example 1 were changed to 2 wt % Triton X-100 and 5 wt % CATINAL SPC-20V-S plus 1.33 wt % LIPOTHOQUAD O/12 respectively; and only a positive sample having an antigen concentration of 10 ng/mL was prepared as the sample in (6) in Example 1. Here, the developing solution prepared in Example 46 contained tri(polyoxyethylene)stearyl ammonium chloride (5 E.O.) at 1 wt % and oleylbis(2-hydroxyethyl)methylammonium chloride at 1 wt %.

### Example 47

This Example was performed in the same manner as Example 1 except as follows: 1.5 wt % Triton X-100 and 5 wt % CATINAL SPC-20V-S in (5) in Example 1 were changed to 2 wt % Triton X-100 plus 1 wt % Tween 20 and 5 wt % CATINAL SPC-20V-S plus 1.33 wt % LIPOTHOQUAD C/12 respectively; and only a positive sample having an antigen concentration of 10 ng/ml was prepared as the sample in (6) in Example 1. Here, the developing solution prepared in Example 47 contained tri(polyoxyethylene)stearyl ammonium chloride (5 E.O.) at 1 wt % and cocoalkylbis(2-hydroxyethyl)methylammonium chloride at 1 wt %.

### Example 48

This Example was performed in the same manner as Example 1 except as follows: 1.5 wt % Triton X-100 in (5) in Example 1 was changed to 2 wt % Triton X-100 plus 1 wt % EMULGEN 150; and only a positive sample having an antigen concentration of 10 ng/ml was prepared as the sample in (6) in Example 1.

Table 11 shows: the surfactants used to prepare the developing solutions in Examples 37 to 48; and the evaluation results of the detecting capability. In Table 11, the HLB value in a case where two kinds of nonionic surfactants were used is the weighted-average HLB value calculated from the HLB values and amounts of the nonionic surfactants. In the same manner, the HLB value in a case where two kinds of cationic surfactants were used is the weighted-average HLB value calculated from the HLB values and amounts of the cationic surfactants.

TABLE 11

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Example 37 | Triton X-100 (2 wt %) | 13.4 | cocoalkylbis(2-hydroxyethyl) | 25.8 | 492.5 |
| | | | methylammonium chloride | | |
| | | | (1 wt %) | | |
| Example 38 | Triton X-100 (2 wt %) | 13.4 | cocoalkylbis(2-hydroxyethyl) | 25.8 | 505.1 |
| | | | methylammonium chloride | | |
| | | | (1.5 wt %) | | |
| Example 39 | Triton X-100 (2 wt %) | 14.5 | cocoalkylbis(2-hydroxyethyl) | 25.8 | 486.4 |
| | Tween 20 (1 wt %) | | methylammonium chloride | | |
| | | | (1.5 wt %) | | |
| Example 40 | Triton X-100 (2 wt %) | 15.1 | cocoalkylbis(2-hydroxyethyl) | 25.8 | 507.5 |
| | EMULGEN 150 (1 wt %) | | methylammonium chloride | | |
| | | | (1.5 wt %) | | |
| Example 41 | Triton X-100 (2 wt %) | 13 | cocoalkylbis(2-hydroxyethyl) | 25.8 | 498.0 |
| | EMULGEN 108 (1 wt %) | | methylammonium chloride | | |
| | | | (1.5 wt %) | | |
| Example 42 | Triton X-100 (2 wt %) | 14.6 | cocoalkylbis(2-hydroxyethyl) | 25.8 | 515.4 |
| | Brij 35 (1 wt %) | | methylammonium chloride | | |
| | | | (1.5 wt %) | | |
| Example 43 | Triton X-100 (2 wt %) | 12.5 | cocoalkylbis(2-hydroxyethyl) | 25.8 | 468.6 |
| | AMIET 105A (1 wt %) | | methylammonium chloride | | |
| | | | (1.5 wt %) | | |
| Example 44 | Triton X-100 (2 wt %) | 14.1 | cocoalkylbis(2-hydroxyethyl) | 25.8 | 492.5 |
| | AMIET320 (1 wt %) | | methylammonium chloride | | |
| | | | (1.5 wt %) | | |
| Example 45 | Triton X-100 (2 wt %) | 13.4 | tri(polyoxyethylene) | 25.6 | 489.2 |
| | | | stearylammonium chloride | | |
| | | | (5 E.O.) (1 wt %) | | |
| | | | cocoalkylbis(2-hydroxyethyl) | | |
| | | | methylammonium chloride | | |
| | | | (1 wt %) | | |
| Example 46 | Triton X-100 (2 wt %) | 13.4 | tri(polyoxyethylene) | 24.4 | 437.8 |
| | | | stearylammonium chloride | | |
| | | | (5 E.O.) (1 wt %) | | |
| | | | oleylbis(2-hydroxyethyl) | | |
| | | | methylammonium chloride | | |
| | | | (1 wt %) | | |
| Example 47 | Triton X-100 (2 wt %) | 14.5 | tri(polyoxyethylene) | 25.6 | 468.7 |
| | Tween 20 (1 wt %) | | stearylammonium chloride | | |
| | | | (5 E.O.) (1 wt %) | | |
| | | | cocoalkylbis(2-hydroxyethyl) | | |
| | | | methylammonium chloride | | |
| | | | (1 wt %) | | |
| Example 48 | Triton X-100 (2 wt %) | 15.1 | tri(polyoxyethylene) | 25.4 | 513.3 |
| | EMULGEN 150 (1 wt %) | | stearylammonium chloride | | |
| | | | (5 E.O.) (1 wt %) | | |

As shown in Table 11, the developing solution in any of Examples 37 and 48 exhibited a high color development intensity. Accordingly, it has been revealed that, as each of the alkylene oxide-addition cationic surfactant and nonionic surfactant contained in the developing solution, one kind of or two or more kinds of such surfactant(s) may be used.

### Example 49

This Example was performed in the same manner as Example 1 except as follows: only a positive sample having an antigen concentration of 10 ng/mL was prepared as the sample in (6) in Example 1; and 75 μL of the sample was dropped onto a sample pad, after which the color development intensity of the test line was measured every 30 seconds using an immunochromato reader until 15 minutes elapsed.

### Example 50

This Example was performed in the same manner as Example 1 except as follows: N102 (MPC (2-methacryloyloxyethylphosphorylcholine) polymer, manufactured by NOF Corporation) was added at 10 v/v % in (5) in Example 1; only a positive sample having an antigen concentration of 10 ng/mL was prepared as the sample in (6) in Example 1; and 75 μL of the sample was dropped onto a sample pad, after which the color development intensity of the test line was measured every 30 seconds using an immunochromato reader until 15 minutes elapsed.

### Example 51

This Example was performed in the same manner as Example 1 except as follows: polyethylene glycol (having an average molecular weight of 4,000) was added at 1 wt % in (5) in Example 1; only a positive sample having an antigen concentration of 10 ng/mL was prepared as the sample in (6) in Example 1; and 75 μL of the sample was dropped onto a sample pad, after which the color development intensity of the test line was measured every 30 seconds using an immunochromato reader until 15 minutes elapsed.

### Example 52

This Example was performed in the same manner as Example 1 except as follows: polyvinylpyrrolidone (having an average molecular weight of 40,000) was added at 1 wt % in (5) in Example 1; only a positive sample having an antigen concentration of 10 ng/mL was prepared as the sample in (6) in Example 1; and 75 μL of the sample was dropped onto a sample pad, after which the color development intensity of the test line was measured every 30 seconds using an immunochromato reader until 15 minutes elapsed.

### Example 53

This Example was performed in the same manner as Example 1 except as follows: polyvinyl alcohol (having a degree of polymerization of 500) was added at 1 wt % in (5) in Example 1; only a positive sample having an antigen concentration of 10 ng/mL was prepared as the sample in (6) in Example 1; and 75 μL of the sample was dropped onto a sample pad, after which the color development intensity of the test line was measured every 30 seconds using an immunochromato reader until 15 minutes elapsed.

### Example 54

This Example was performed in the same manner as Example 1 except as follows: poly(2-ethyl-2-oxazoline) (having an average molecular weight of 50,000) was added at 1 wt % in (5) in Example 1; only a positive sample having an antigen concentration of 10 ng/ml was prepared as the sample in (6) in Example 1; and 75 μL of the sample was dropped onto a sample pad, after which the color development intensity of the test line was measured every 30 seconds using an immunochromato reader until 15 minutes elapsed.

### Example 55

This Example was performed in the same manner as Example 1 except as follows: dextran (having an average molecular weight of 40,000) was added at 1 wt % in (5) in Example 1; only a positive sample having an antigen concentration of 10 ng/mL was prepared as the sample in (6) in Example 1; and 75 μL of the sample was dropped onto a sample pad, after which the color development intensity of the test line was measured every 30 seconds using an immunochromato reader until 15 minutes elapsed.

### Example 56

This Example was performed in the same manner as Example 1 except as follows: bovine serum albumin was added at 1 wt % in (5) in Example 1; only a positive sample having an antigen concentration of 10 ng/mL was prepared as the sample in (6) in Example 1; that 75 μL of the sample was dropped onto a sample pad, after which the color development intensity of the test line was measured every 30 seconds using an immunochromato reader until 15 minutes elapsed.

### Example 57

This Example was performed in the same manner as Example 1 except as follows: casein was added at 1 wt % in (5) in Example 1; only a positive sample having an antigen concentration of 10 ng/ml was prepared as the sample in (6) in Example 1; and 75 μL of the sample was dropped onto a sample pad, after which the color development intensity of the test line was measured every 30 seconds using an immunochromato reader until 15 minutes elapsed.

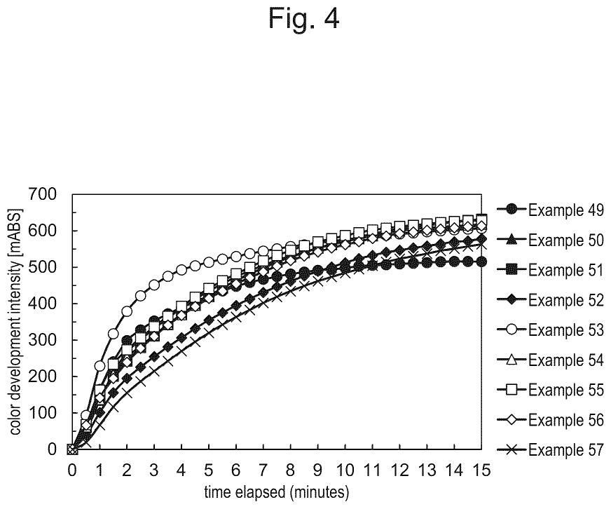

Table 12 shows: the additives (stabilizing components) used in Examples 49 to 57; and the evaluation results of the detecting capability after the elapse of 15 minutes. In addition, FIG. 4 illustrates the relationship between the color development intensity of a positive sample and the time elapsed, wherein the sample was produced in each of Examples 49 to 57.

TABLE 12

| | | Color Development |
|---|---|---|
| | | Intensity [mABS] |
| | | Antigen |
| | | Concentration [ng/mL] |
| | Additive | 10 |
| Example 49 | — | 515.2 |
| Example 50 | N102(10 v/v %) | 630.0 |
| Example 51 | polyethylene glycol (average molecular weight, 4,000) (1 wt %) | 631.4 |
| Example 52 | polyvinylpyrrolidone (average molecular weight, 40,000) (1 wt %) | 578.1 |
| Example 53 | polyvinyl alcohol (degree of polymerization, 500) (1 wt %) | 606.6 |
| Example 54 | poly(2-ethyl-2-oxazoline) (average molecular weight, 50,000) (1 wt %) | 617.8 |
| Example 55 | dextran (average molecular weight, 40,000) (1 wt %) | 627.2 |
| Example 56 | bovine serum albumin (1 wt %) | 613.1 |
| Example 57 | casein (1 wt %) | 562.8 |

As shown in Table 12, the developing solutions in Examples 49 to 57 exhibited a high color development intensity when 15 minutes elapsed after the positive sample was dropped onto the sample pad. The developing solution containing an additive (stabilizing component) tended to exhibit a higher color development intensity than the developing solution containing no additive (stabilizing component). The above results have revealed that some kind of additive may be used as a component of the developing solution.

FIG. 4 revealed that the color development intensity of the developing solution in each of Examples 49 to 57 was rapidly increased after the positive sample was dropped onto the sample pad. When 30 seconds elapsed after the positive sample was dropped, Examples 49 to 57 exhibited a color development intensity of 15 mABS or more, enabling the coloration of the test line to be visually recognized. As described above, the chromatographic developing solution including an alkylene oxide-addition cationic surfactant, a nonionic surfactant, and some kind of additive achieved high-sensitivity chromatography.

### Example 58

This Example was performed in the same manner as Example 1 except (6) in Example 1 was changed as described below.

### (6) Evaluation of Detecting Capability

To the developing solution, a heat-inactivated SARS-COV-2 virus antigen (ATCC No. VR-1986HK, 2019-nCoV/USA-WA1/2020) was added at 6.45×10⁵ TCID₅₀/mL to prepare a positive sample.

Onto the sample pad, 75 μL of the sample was dropped, and the color development intensity of the test line was measured every 30 seconds using an immunochromato reader until 15 minutes elapsed.

### Comparative Example 25

This Comparative Example was performed in the same manner as Example 1 except: 5 wt % CATINAL SPC-20V-S in (5) in Example 1 was not used; and (6) in Example 1 was changed to (6) in Example 58.

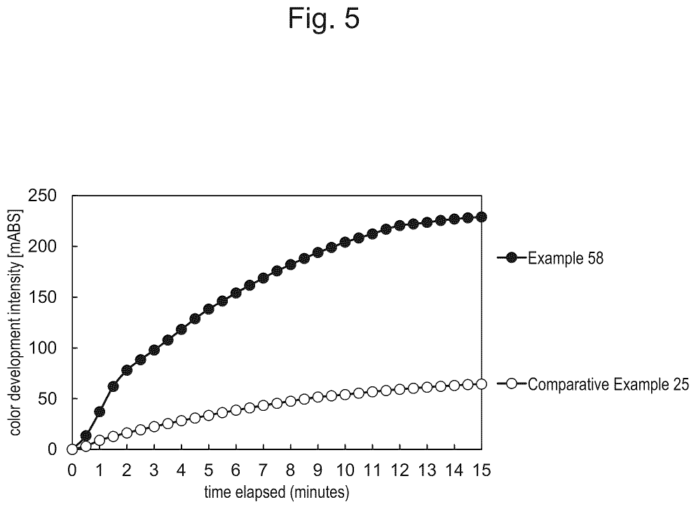

Table 13 shows: the surfactants used to prepare the developing solutions in Example 58 and Comparative Example 25; and the evaluation results of the detecting capability after the elapse of 15 minutes. In addition, FIG. 5 illustrates the relationship between the color development intensity of a positive sample and the time elapsed, wherein the sample was produced in each of Example 58 and Comparative Example 25.

TABLE 13

| | | | | | Color Development |
|---|---|---|---|---|---|
| | | | | | Intensity [mABS] |
| | Surfactant | | | | Antigen Concentration |
| | Nonionic | HLB | Cationic | HLB | [TCID50/mL] |
| | Surfactant | (Griffin) | Surfactant | (Davies) | 6.45 × 10\hat{ }5 |
| Example 58 | Triton X-100 | 13.4 | tri(polyoxyethylene) | 25.4 | 229.0 |
| | (1.5 wt %) | | stearylammonium chloride | | |
| | | | (5 E.O.) (1 wt %) | | |
| Comparative | Triton X-100 | 13.4 | — | | 64.4 |
| Example 25 | (1.5 wt %) | | | | |

Table 13 and FIG. 5 revealed that, in Example 58, the color development intensity was larger than in Comparative Example 25. As the evaluation of the use of a virus antigen has verified, the use affords the same effect as the use of a nucleocapsid protein (NP) antigen.

### Example 59

This Example was performed in the same manner as Example 1 except (2), (4), (5), and (6) in Example 1 were changed as described below.

### (2) Production of Anti-SARS-COV-2 NP Antibody-Bound Gold Colloid-Coated Conjugate Pad

An antibody-bound gold colloid coating liquid was prepared by the method described in (2) in Example 1.

The antibody-bound gold colloid coating liquid was uniformly applied at 0.5 μL/mm² to a glass fiber pad cut out in a shape 15 mm high and 300 mm long.

Then, the glass fiber pad coated with the antibody-bound gold colloid coating liquid was dried with a vacuum dryer to obtain a conjugate pad.

### (4) Production of Immunochromatographic Device

To a polypropylene backing sheet (manufactured by Lohmann Tape Group) as a base material, the conjugate pad produced in (2) above, the antibody-coated membrane produced by the method described (3) in Example 1, a glass fiber sample pad, and a cellulose absorption pad were bonded in such a manner that the sample pad, the conjugate pad, the antibody-coated membrane, and the absorption pad were superposed one on another in this order from upstream. Using a cutting machine, the resulting product was cut to a width of 4 mm to obtain a test piece 4 mm wide and 75 mm long, which was encapsulated in a housing case (manufactured by Shin Corporation) to produce an immunochromatographic device.

### (5) Preparation of Developing Solution

To a 5 mM citrate buffer, Triton X-100 as a nonionic surfactant and LIPOTHOQUAD C/12 as a cationic surfactant were added at 2 wt % and 1.33 wt % respectively, and sodium chloride was further added at 150 mM to prepare a developing solution. The resulting developing solution contained cocoalkylbis(2-hydroxyethyl)methylammonium chloride at 1 wt %.

### (6) Evaluation of Detecting Capability

A cotton swab (FLOQ swab 534100CS01-E, manufactured by Copan Italia SpA) that had collected a SARS-COV-2-negative liquid wiped off the nasopharynx was put into 400 μL of the developing solution, and the liquid wiped off the nasopharynx was thus suspended in the developing solution to prepare a negative sample.

To the negative sample, the SARS-COV-2 NP antigen was added at 1 ng/ml to prepare a positive sample.

When 15 minutes elapsed after 75 μL of the sample was dropped onto the sample pad, the color development intensity of the test line was measured using an immunochromato reader.

### Example 60

This Example was performed in the same manner as Example 59 except 1.33 wt % LIPOTHOQUAD C/12 in (5) in Example 59 was changed to 2 wt % LIPOTHOQUAD C/12. Here, the developing solution prepared in Example 60 contains cocoalkylbis(2-hydroxyethyl)methylammonium chloride at 1.5 wt %.

### Example 61

This Example was performed in the same manner as Example 59 except as follows: 1.33 wt % LIPOTHOQUAD C/12 in (5) Example 59 was changed to 2 wt % LIPOTHOQUAD C/12; and, as nonspecific-adsorption inhibitors, L-arginine hydrochloride and ethylenediaminetetraacetic acid disodium were added at 0.2 wt % and 10 mM respectively. Here, the developing solution prepared in Example 61 contains cocoalkylbis(2-hydroxyethyl)methylammonium chloride at 1.5 wt %.

### Comparative Example 26

This Comparative Example was performed in the same manner as Example 59 except 1.33 wt % LIPOTHOQUAD C/12 in (5) in Example 59 was not used.

Table 14 shows: the surfactants and additives (excluding a buffer and sodium chloride) used to prepare the developing solutions in Examples 59 and 61 and Comparative Example 26; and the evaluation results of the detecting capability.

TABLE 14

| | | | | | | Color Development | |
|---|---|---|---|---|---|---|---|
| | | | | | | Intensity [mABS] | |
| | Surfactant | | | | | Antigen | |
| | Nonionic | HLB | Cationic | HLB | | Concentration [ng/mL] | |
| | Surfactant | (Griffin) | Surfactant | (Davies) | Additive | 0 | 1 |
| Example 59 | Triton X-100 | 13.4 | cocoalkylbis | 25.8 | — | N.D. | 184.2 |
| | (2 wt %) | | (2-hydroxyethyl) | | | | |
| | | | methylammonium | | | | |
| | | | chloride (1 wt %) | | | | |
| Example 60 | Triton X-100 | 13.4 | cocoalkylbis | 25.8 | — | N.D. | 127.0 |
| | (2 wt %) | | (2-hydroxyethyl) | | | | |
| | | | methylammonium | | | | |
| | | | chloride (1.5 wt %) | | | | |
| Example 61 | Triton X-100 | 13.4 | cocoalkylbis | 25.8 | 0.2 wt % L-ARGININE | N.D. | 82.5 |
| | (2 wt %) | | (2-hydroxyethyl) | | HYDROCHLORIDE 10 mM | | |
| | | | methylammonium | | ethylenediaminetetraacetic | | |
| | | | chloride (1.5 wt %) | | acid disodium | | |
| Comparative | Triton X-100 | 13.4 | — | | — | N.D. | 13.5 |
| Example 26 | (2 wt %) | | | | | | |

It has been revealed that Examples 59 to 61 afforded a larger color development intensity than Comparative Example 26. It has been verified that even the sample having thereto liquid wiped off the nasopharynx afforded the same effect as the sample containing a nucleocapsid protein (NP) antigen. In Example 60, the residual coloration of the gold colloid was slightly observed in the background (portion other than the test line and the control line in the membrane), but the coloration of the background was slightly inhibited in Example 61 performed using the nonspecific-adsorption inhibitor, and the visibility of the coloration of the test line and the control line was enhanced.

### Example 62

An immunochromatographic device was produced in the same manner as in (1) to (4) in Example 1 except the glass fiber sample pad in (4) in Example 1 was changed to a pretreated sample pad obtained in (A) below.

### (A) Production of Pretreated Sample Pad

To a 5 mM citrate buffer, CATINAL SPC-20V-S was added at 5 wt %, and sodium chloride was further added at 150 mM to prepare a sample pad coating liquid. The resulting sample pad coating liquid contained tri(polyoxyethylene)stearyl ammonium chloride (5 E.O.) at 1 wt %.

The sample pad coating liquid was uniformly applied at 0.5 μL/mm² to the glass fiber sample pad.

Then, the sample pad coated with the sample pad coating liquid was dried with a vacuum dryer to obtain a pretreated sample pad.

### (5) Preparation of Developing Solution

To a 5 mM citrate buffer, Triton X-100 was added at 1.5 wt %, and sodium chloride was further added at 150 mM to prepare a developing solution.

### (6) Evaluation of Detecting Capability

To the developing solution, the SARS-COV-2 NP antigen was added at 10 ng/ml to prepare a positive sample.

When 15 minutes elapsed after 75 μL of the sample was dropped onto the sample pad, the color development intensity of the test line was measured using an immunochromato reader.

### Example 63

This Example was performed in the same manner as Example 62 except Triton X-100 was added at 1.5 wt % to the sample pad coating liquid in (A) in Example 62.

### Comparative Example 27

This Comparative Example was performed in the same manner as Example 62 except 5 wt % CATINAL SPC-20V-S was not used in (A) in Example 62.

### Comparative Example 28

This Comparative Example was performed in the same manner as Example 62 except 5 wt % CATINAL SPC-20V-S was changed to 1.5 wt % Triton X-100 in (A) in Example 62.

Table 15 shows: the surfactants used to prepare the sample pad coating liquids in Examples 62 and 63 and Comparative Examples 27 to 28; and the evaluation results of the detecting capability.

TABLE 15

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Example 62 | — | | tri(polyoxyethylene) | 25.4 | 199.8 |
| | | | stearylammonium chloride | | |
| | | | (5 E.O.) (1 wt %) | | |
| Example 63 | Triton X-100 | 13.4 | tri(polyoxyethylene) | 25.4 | 150.2 |
| | (1.5 wt %) | | stearylammonium chloride | | |
| | | | (5 E.O.) (1 wt %) | | |
| Comparative | — | | — | | 66.6 |
| Example 27 | | | | | |
| Comparative | Triton X-100 | 13.4 | — | | 92.5 |
| Example 28 | (1.5 wt %) | | | | |

It has been revealed that Examples 62 to 63 afforded a larger color development intensity than Comparative Examples 27 and 28. This is considered to be because, in Examples, the developing solution contained a nonionic surfactant, the sample pad contained at least an alkylene oxide-addition cationic surfactant, and thus, in the whole, the nonionic surfactant and the alkylene oxide-addition cationic surfactant were present.

### Example 64

An immunochromatographic device was produced in the same manner as in (1) to (4) in Example 1 except (2) in Example 1 was changed as described below.

### (2) Production of Anti-SARS-COV-2 NP Antibody-Bound Gold Colloid-Coated Conjugate Pad

To the antibody-bound gold colloid suspension produced in (1) above, sucrose, polyethylene glycol (having an average molecular weight of 20,000), and bovine serum albumin were added at 5 wt %, 0.05 wt %, and 1 wt % respectively, and Triton X-100 and CATINAL SPC-20V-S were further added at 0.5 wt % and 1.65 wt % respectively to prepare an antibody-bound gold colloid coating liquid. The resulting antibody-bound gold colloid coating liquid contained tri(polyoxyethylene)stearyl ammonium chloride (5 E.O.) at 0.33 wt %.

The antibody-bound gold colloid coating liquid was uniformly applied at 0.5 L/mm² to a glass fiber pad cut out in a shape 7 mm high and 300 mm long.

Then, the glass fiber pad coated with the antibody-bound gold colloid coating liquid was dried with a vacuum dryer to obtain a conjugate pad.

### (5) Preparation of Developing Solution

To a 5 mM citrate buffer, Triton X-100 was added at 1.5 wt %, and sodium chloride was further added at 150 mM to prepare a developing solution.

### (6) Evaluation of Detecting Capability

To the developing solution, the SARS-COV-2 NP antigen was added at 10 ng/ml to prepare a positive sample.

When 15 minutes elapsed after 75 μL of the sample was dropped onto the sample pad, the color development intensity of the test line was measured using an immunochromato reader.

### Comparative Example 29

This Comparative Example was performed in the same manner as Example 64 except neither 0.5 wt % Triton X-100 nor 1.65 wt % CATINAL SPC-20V-S was used in (2) in Example 64.

### Comparative Example 30

This Comparative Example was performed in the same manner as Example 64 except 1.65 wt % CATINAL SPC-20V-S was not used in (2) in Example 64.

Table 16 shows: the surfactants used to prepare the antibody-bound gold colloid coating liquids in Example 64 and Comparative Examples 29 and 30; and the evaluation results of the detecting capability.

TABLE 16

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Example 64 | Triton X-100 | 13.4 | tri(polyoxyethylene) | 25.4 | 107.3 |
| | (0.5 wt %) | | stearylammonium chloride | | |
| | | | (5 E.O.) (0.33 wt %) | | |
| Comparative | — | | — | | 73.0 |
| Example 29 | | | | | |
| Comparative | Triton X-100 | 13.4 | — | | 49.0 |
| Example 30 | (0.5 wt %) | | | | |

It has been revealed that Example 64 afforded a larger color development intensity than Comparative Examples 29 and 30. This is considered to be because, in Examples, the developing solution contained a nonionic surfactant, the conjugate pad contained a nonionic surfactant and an alkylene oxide-addition cationic surfactant, and thus, in the whole, the nonionic surfactant and the alkylene oxide-addition cationic surfactant were present.

### Example 65

This Example was performed in the same manner as Example 1 except as follows: (1) and (2) in Example 1 were changed as described below; in (3) in Example 1, the nitrocellulose membrane (HF120, manufactured by Merk Millipore) was changed to a nitrocellulose membrane (HF075, manufactured by Merk Millipore); and only a positive sample having an antigen concentration of 10 ng/mL was prepared as the sample in (6) in Example 1.

### (1) Production of Anti-SARS-COV-2 NP Antibody-Bound Colored Latex

To 0.5 mL of a carboxyl group-modified red polystyrene latex solution (having a particle diameter of 400 nm and a concentration of 10 w/v %; manufactured by MagSphere Inc.), 4.5 mL of sterile distilled water was added, and the resulting mixture was stirred.

The mixture was centrifuged at 10° C. at 15,000 rpm for 15 minutes, the supernatant was removed, and then, the resulting precipitate was suspended in a 30 mM MES (2-Morpholinoethanesulfonic acid, monohydrate) buffer having a pH of 5.8 to obtain a latex suspension.

With 1.15 mL of the latex suspension, 0.2 mL of an aqueous solution of 0.1 wt % Triton X-100 and 0.15 mL of an aqueous solution of a 1 mg/mL mouse anti-SARS-COV-2 NP monoclonal antibody were mixed, and then, the resulting mixture was stirred at 37° C. for 60 minutes.

With the mixture, 0.1 mL of an aqueous solution of 0.5% EDC (N-(3-dimethylaminopropyl)-N′-ethylcarbodiimide hydrochloride) and 0.2 mL of an aqueous solution of 0.5% NHS (N-Hydroxysuccinimide) were further mixed, and then, the resulting mixture was stirred at 37° C. for 60 minutes.

Then, the mixture was centrifuged at 10° C. at 15,000 rpm for 15 minutes, and the supernatant was removed. Then, to the resulting precipitate, a solution containing a 20 mM tris hydrochloric acid buffer having a pH of 8.2, 0.05 wt % polyethylene glycol (having an average molecular weight of 20,000), and 1 wt % bovine serum albumin was added to suspend the precipitate. This buffer replacement was performed again to obtain an anti-SARS-COV-2 NP antibody-bound colored latex solution.

### (2) Production of Anti-SARS-COV-2 NP Antibody-Bound Colored Latex-Coated Conjugate Pad

To the antibody-bound colored latex solution produced in (1) above, sucrose and Triton X-100 were added at 5 wt % and 0.5 wt % respectively to prepare an antibody-bound colored latex coating liquid.

The antibody-bound colored latex coating liquid was uniformly applied at 0.5 L/mm² to a glass fiber pad cut out in a shape 7 mm high and 300 mm long.

Then, the glass fiber pad coated with the antibody-bound colored latex coating liquid was dried with a vacuum dryer to obtain a conjugate pad.

### Comparative Example 31

This Comparative Example was performed in the same manner as Example 65 except 5 wt % CATINAL SPC-20V-S in (5) in Example 65 was not used.

### Comparative Example 32

This Comparative Example was performed in the same manner as Example 65 except 1.5 wt % Triton X-100 in (5) in Example 65 was not used.

### Comparative Example 33

This Comparative Example was performed in the same manner as Example 65 except 5 wt % CATINAL SPC-20V-S in (5) in Example 65 was changed to 1 wt % CTAC (cetyltrimethylammonium chloride).

Table 17 shows: the surfactants used to prepare the developing solutions in Example 65 and Comparative Examples 31 to 33; and the evaluation results of the detecting capability.

TABLE 17

| | | | | | Color Development |
|---|---|---|---|---|---|
| | | | | | Intensity [mABS] |
| | Surfactant | | | | Antigen |
| | Nonionic | HLB | Cationic | HLB | Concentration [ng/mL] |
| | Surfactant | (Griffin) | Surfactant | (Davies) | 10 |
| Example 65 | Triton X-100 | 13.4 | tri(polyoxyethylene) | 25.4 | 101.5 |
| | (1.5 wt %) | | stearylammonium chloride | | |
| | | | (5 E.O.) (1 wt %) | | |
| Comparative | Triton X-100 | 13.4 | — | | 3.4 |
| Example 31 | (1.5 wt %) | | | | |
| Comparative | — | | tri(polyoxyethylene) | 25.4 | Invalid incomplete |
| Example 32 | | | stearylammonium chloride | | color development |
| | | | (5 E.O.) (1 wt %) | | in Line |
| Comparative | Triton X-100 | 13.4 | cetyltrimethylammonium | | N.D. |
| Example 33 | (1.5 wt %) | | chloride (1 wt %) | | |

It has been revealed that Example 65 afforded a larger color development intensity than Comparative Examples 31 to 33. It has been verified that using an antibody-bound latex afforded the same effect as using an antibody-bound gold colloid.

### Example 66

### (1) Production of Antibody-Bound Gold Colloid

This Example was performed in the same manner as (1) in Example 1 except the mouse anti-SARS-COV-2 NP monoclonal antibody in (1) in Example 1 was changed to a mouse anti-influenza A virus NP monoclonal antibody to obtain an anti-influenza A virus NP antibody-bound gold colloid suspension (antibody-bound gold colloid suspension).

This Example was performed in the same manner as (1) in Example 1 except the mouse anti-SARS-COV-2 NP monoclonal antibody in (1) in Example 1 was changed to a mouse anti-influenza B virus NP monoclonal antibody to obtain an anti-influenza B virus NP antibody-bound gold colloid suspension (antibody-bound gold colloid suspension).

A suspension of an anti-SARS-COV-2 NP antibody-bound gold colloid (an antibody-bound gold colloid suspension) was obtained in the same manner as in (1) in Example 1.

That is, three kinds of antibody-bound gold colloid suspensions were prepared in Example 66.

### (2) Production of Antibody-Bound Gold Colloid-Coated Conjugate Pad

To each of the three kinds of antibody-bound gold colloid suspensions produced in (1) above, sucrose, polyethylene glycol (having an average molecular weight of 20,000), and bovine serum albumin were added at 5 wt %, 0.05 wt %, and 1 wt % respectively to prepare three kinds of antibody-bound gold colloid coating liquids.

A coating liquid obtained by mixing the three kinds of antibody-bound gold colloid coating liquids in equivalent amounts was uniformly applied at 0.5 μL/mm² to a glass fiber pad cut out in a shape 10 mm high and 300 mm long.

Then, the glass fiber pad coated with the antibody-bound gold colloid coating liquid was dried with a vacuum dryer to obtain a conjugate pad.

### (3) Production of Antibody-Coated Membrane

To a 12 mm high position of a nitrocellulose membrane (HF120, manufactured by Merk Millipore) cut out in a shape 30 mm high and 300 mm long, a solution containing a 1 mg/mL mouse anti-influenza A NP monoclonal antibody and 2.5 wt % sucrose in a 5 mM phosphate buffer was applied, using a dispenser, at 1 μL/cm in the form of a line 1 mm wide and perpendicular to the direction of development to produce an influenza A test line (A line).

To a 15 mm high position of the nitrocellulose membrane, a solution containing 1 mg/mL mouse anti-influenza B NP monoclonal antibody and 2.5 wt % sucrose in a 5 mM phosphate buffer was applied, using a dispenser, at 1 μL/cm in the form of a line 1 mm wide and perpendicular to the direction of development to produce an influenza B test line (B line).

To an 18 mm high position of the nitrocellulose membrane, a solution containing a 1 mg/mL mouse anti-SARS-COV-2 NP monoclonal antibody and 2.5 wt % sucrose in a 5 mM phosphate buffer was applied, using a dispenser, at 1 μL/cm in the form of a line 1 mm wide and perpendicular to the direction of development to produce a SARS-COV-2 test line (S line).

To a 21 mm high position of the nitrocellulose membrane, a solution containing 1 mg/mL goat anti-mouse immunoglobulin polyclonal antibody and 2.5 wt % sucrose in a 5 mM phosphate buffer was applied, using a dispenser, at 1 μL/cm in the form of a line 1 mm wide and perpendicular to the direction of development to produce a control line (C line).

Then, the nitrocellulose membrane with the test line and the control line produced by coating was dried using a vacuum dryer to obtain an antibody-coated membrane.

### (4) Production of Immunochromatographic Device

To a polypropylene backing sheet (manufactured by Lohmann Tape Group) as a base material, the conjugate pad produced in (2) above, the antibody-coated membrane produced in (3) above, a glass fiber sample pad, and a cellulose absorption pad were bonded in such a manner that the sample pad, the conjugate pad, the antibody-coated membrane, and the absorption pad were superposed one on another in this order from upstream. Using a cutting machine, the resulting product was cut to a width of 4 mm to produce an immunochromatographic device 4 mm wide and 60 mm long.

### (5) Preparation of Developing Solution

To phosphate buffered saline (PBS), Triton X-100 as a nonionic surfactant and CATINAL SPC-20V-S as a cationic surfactant were added at 1.5 wt % and 5 wt % respectively to prepare a developing solution. The resulting developing solution contained tri(polyoxyethylene)stearyl ammonium chloride (5 E.O.) at 1 wt %.

### (6) Evaluation of Detecting Capability

To the developing solution, an inactivated influenza A virus antigen (1.86 mg/mL, manufactured by Bio-Rad Laboratories, Inc.) was added to obtain a 1 mg/mL positive sample. The developing solution was further added for dilution in 10-fold steps to prepare an influenza A virus positive sample (having an antigen concentration of 10 μg/mL).

To the developing solution, an inactivated influenza B virus antigen (1.5 mg/mL, manufactured by Bio-Rad Laboratories, Inc.) was added to obtain a 1 mg/mL positive sample. The developing solution was further added for dilution in 10-fold steps to prepare an influenza B virus positive sample (having an antigen concentration of 1 μg/mL).

To a PBS solution of a SARS-COV-2 NP antigen (1 mg/mL), the developing solution was added for dilution in 10-fold steps to prepare a SARS-COV-2 positive sample (having an antigen concentration of 1 ng/ml).

When 15 minutes elapsed after 75 μL of each positive sample was dropped onto the sample pad, the color development intensities of the test line and the control line were measured using an immunochromato reader (C10066, manufactured by Hamamatsu Photonics K.K.).

### Example 67

This Example was performed in the same manner as Example 66 except 5 wt % CATINAL SPC-20V-S in (5) in Example 66 was changed to 1.33 wt % LIPOTHOQUAD C/12. Here, the developing solution prepared in Example 67 contained cocoalkylbis(2-hydroxyethyl)methylammonium chloride at 1 wt %.

### Example 68

This Example was performed in the same manner as Example 66 except (5) in Example 66, CHAPS (3-[(3-cholamido-propyl)dimethylammonio]-1-propanesulfonate) was added at 0.1 wt %.

### Comparative Example 34

This Comparative Example was performed in the same manner as Example 66 except CATINAL SPC-20V-S in (5) in Example 66 was not used.

### Comparative Example 35

This Comparative Example was performed in the same manner as Example 66 except 5 wt % CATINAL SPC-20V-S in (5) in Example 66 was changed to 1 wt % STAC (stearyltrimethylammonium chloride).

### Comparative Example 36

This Comparative Example was performed in the same manner as Example 66 except 5 wt % CATINAL SPC-20V-S in (5) in Example 66 was changed to 1 wt % SDS (sodium dodecylsulfate).

Tables 18 and 19 show: the surfactants used to prepare the developing solutions in Examples 66 to 68 and Comparative Examples 34 to 36; and the evaluation results of the detecting capability.

TABLE 18

| | Surfactant | | | |
|---|---|---|---|---|
| | Nonionic | Cationic | Anionic | Ampholytic |
| | Surfactant | Surfactant | Surfactant | Surfactant |
| Example 66 | Triton X-100 | tri(polyoxyethylene) | — | — |
| | (1.5 wt %) | stearylammonium chloride | | |
| | | (5 E.O.) (1 wt %) | | |
| Example 67 | Triton X-100 | cocoalkylbis(2-hydroxyethyl) | — | — |
| | (1.5 wt %) | methylammonium chloride | | |
| | | (1 wt %) | | |
| Example 68 | Triton X-100 | tri(polyoxyethylene) | — | CHAPS |
| | (1.5 wt %) | stearylammonium chloride | | (0.1 wt %) |
| | | (5 E.O.) (1 wt %) | | |
| Comparative | Triton X-100 | — | — | — |
| Example 34 | (1.5 wt %) | | | |
| Comparative | Triton X-100 | stearyltrimethylammonium | — | — |
| Example 35 | (1.5 wt %) | chloride (1 wt %) | | |
| Comparative | Triton X-100 | — | sodium | — |
| Example 36 | (1.5 wt %) | | dodecylsulfate (1 wt %) | |

TABLE 19

| | Color Development Intensity [mABS] | | | | | | | | | | | |
|---|---|---|---|---|---|---|---|---|---|---|---|---|
| | Inactivated Influenza | | | | Inactivated Influenza | | | | SARS-CoV-2 NP | | | |
| | A Virus Antigen | | | | B Virus Antigen | | | | Antigen Concentration: | | | |
| | Concentration: 10 μg/mL | | | | Concentration: 1 μg/mL | | | | 1 ng/mL | | | |
| | C Line | S Line | B Line | A Line | C Line | S Line | B Line | A Line | C Line | S Line | B Line | A Line |
| Example 66 | 637.0 | N.D. | N.D. | 104.6 | 451.4 | N.D. | 206.0 | N.D. | 501.5 | 290.4 | N.D. | N.D. |
| Example 67 | 597.9 | N.D. | N.D. | 131.1 | 634.1 | N.D. | 172.3 | N.D. | 572.0 | 295.0 | N.D. | N.D. |
| Example 68 | 692.7 | N.D. | N.D. | 103.4 | 644.7 | N.D. | 211.5 | N.D. | 624.2 | 294.7 | N.D. | N.D. |
| Comparative Example 34 | 504.9 | N.D. | N.D. | 28.7 | 486.0 | N.D. | 138.0 | N.D. | 500.2 | 205.9 | N.D. | N.D. |
| Comparative Example 35 | 489.6 | N.D. | N.D. | 70.9 | 585.7 | N.D. | 136.6 | N.D. | 391.2 | 160.5 | N.D. | N.D. |
| Comparative Example 36 | 276.2 | N.D. | N.D. | N.D. | 231.9 | N.D. | N.D. | N.D. | 288.2 | 3.5 | N.D. | N.D. |

As shown in Table 19, the developing solutions in Examples 66 to 68, compared with Comparative Examples 34 to 36, exhibited a high color development intensity. Any of the test lines corresponding to the respective positive samples were colored, and no test line other than them was colored. As described above, the chromatographic developing solution including an alkylene oxide-addition cationic surfactant and a nonionic surfactant caused no nonspecific reaction in the presence of a plurality of test lines, and achieved high-sensitivity chromatography.

In addition, in a case where the mouse anti-influenza A virus NP monoclonal antibody-bound gold colloid or the mouse anti-influenza B virus NP monoclonal antibody-bound gold colloid was used as a labeling substance, where the mouse anti-influenza A virus NP monoclonal antibody or the mouse anti-influenza B virus NP monoclonal antibody was used as a trapping substance, and where the inactivated influenza A virus antigen or the inactivated influenza B virus antigen was used as an analyte, the chromatographic developing solution containing the alkylene oxide-addition cationic surfactant and the nonionic surfactant achieved high-sensitivity chromatography.

In addition, the developing solution in Example 68 caused no nonspecific reaction, and exhibited almost the same high color development intensity as Example 66. It has been verified that adding an ampholytic surfactant does not affect the color development intensity.

### Example 69

This Example was performed in the same manner as Example 1 except as follows: the mouse anti-SARS-COV-2 NP monoclonal antibody in (1) to (3) in Example 1 was changed to a mouse anti-D dimer monoclonal antibody; the SARS-COV-2 NP antigen in (6) in Example 1 was changed to a D-dimer antigen (D-dimer Calibrator, 60 μg/mL; manufactured by Sekisui Medical Co., Ltd.); and the developing solution was added for dilution to prepare a D-dimer positive sample (having an antigen concentration of 1 μg/mL). Here, the developing solution prepared in Example 69 contains tri(polyoxyethylene)stearyl ammonium chloride (5 E.O.) at 1 wt %.

### Example 70

This Example was performed in the same manner as Example 69 except 5 wt % CATINAL SPC-20V-S in (5) in Example 69 was changed to 1.33 wt % LIPOTHOQUAD C/12. Here, the developing solution prepared in Example 70 contains cocoalkylbis(2-hydroxyethyl)methylammonium chloride at 1 wt %.

### Comparative Example 37

This Comparative Example was performed in the same manner as Example 69 except CATINAL SPC-20V-S in (5) in Example 69 was not used.

### Comparative Example 38

This Comparative Example was performed in the same manner as Example 69 except 5 wt % CATINAL SPC-20V-S in (5) in Example 69 was changed to 1 wt % CTAC (cetyltrimethylammonium chloride).

### Comparative Example 39

This Comparative Example was performed in the same manner as Example 69 except 5 wt % CATINAL SPC-20V-S in (5) in Example 69 was changed to 1 wt % STAC (stearyltrimethylammonium chloride).

### Comparative Example 40

This Comparative Example was performed in the same manner as Example 69 except 5 wt % CATINAL SPC-20V-S in (5) in Example 69 was changed to 1 wt % SDS (sodium dodecylsulfate).

### Comparative Example 41

This Comparative Example was performed in the same manner as Example 69 except 5 wt % CATINAL SPC-20V-S in (5) in Example 69 was changed to 1 wt % SDBS (sodium dodecylbenzenesulfonate).

Table 20 shows: the surfactants used to prepare the developing solutions in Examples 69 and 70 and Comparative Examples 37 to 41; and the evaluation results of the detecting capability.

TABLE 20

| | | | | Color Development | |
|---|---|---|---|---|---|
| | | | | Intensity [mABS] | |
| | | | | D-dimer Antigen | |
| | Surfactant | | | Concentration: | |
| | Nonionic | Cationic | Anionic | 1 μg/mL | |
| | Surfactant | Surfactant | Surfactant | C Line | T Line |
| Example 69 | Triton X-100 | tri(polyoxyethylene) | — | 259.1 | 431.3 |
| | (1.5 wt %) | stearylammonium | | | |
| | | chloride | | | |
| | | (5 E.O.) (1 wt %) | | | |
| Example 70 | Triton X-100 | cocoalkylbis(2-hydroxyethyl) | — | 310.5 | 503.6 |
| | (1.5 wt %) | methylammonium | | | |
| | | chloride (1 wt %) | | | |
| Comparative | Triton X-100 | — | — | 233.2 | 298.0 |
| Example 37 | (1.5 wt %) | | | | |
| Comparative | Triton X-100 | cetyltrimethylammonium | — | 250.1 | 305.6 |
| Example 38 | (1.5 wt %) | chloride (1 wt %) | | | |
| Comparative | Triton X-100 | stearyltrimethylammonium | — | 229.3 | 361.8 |
| Example 39 | (1.5 wt %) | chloride (1 wt %) | | | |
| Comparative | Triton X-100 | — | sodium dodecylsulfate | 193.3 | 1.4 |
| Example 40 | (1.5 wt %) | | (1 wt %) | | |
| Comparative | Triton X-100 | — | sodium | N.D. | 106.8 |
| Example 41 | (1.5 wt %) | | dodecylbenzenesulfonate | | |
| | | | (1 wt %) | | |

As shown in Table 20, the developing solutions in Examples 69 to 70, compared with Comparative Examples 37 to 41, exhibited a high color development intensity. In the case of Comparative Example 41, the developing solution did not even reach the C line. Also in a case where the mouse anti-D dimer monoclonal antibody-bound gold colloid was used as a labeling substance, where the mouse anti-D-dimer monoclonal antibody was used as a trapping substance, and where the D-dimer antigen was used as an analyte, the chromatographic developing solution containing the alkylene oxide-addition cationic surfactant and the nonionic surfactant successfully achieved high-sensitivity chromatography.

In addition, the developing solutions in Examples 66 to 70 exhibited a high color development intensity, thus demonstrating that the chromatographic developing solution containing the alkylene oxide-addition cationic surfactant and the nonionic surfactant successfully achieved high-sensitivity chromatography, regardless of the kind of an antibody or the like used as the trapping substance or the labeling substance and the kind of an antigen or the like regarded as an analyte.

### Example 71

An immunochromatographic device was produced in the same manner as in (1) to (4) in Example 66 except the glass fiber sample pad in (4) in Example 66 was changed to a pretreated sample pad obtained in (A) below.

### (A) Production of Pretreated Sample Pad

A sample pad coating liquid containing 1.66 wt % LIPOTHOQUAD C/12 was prepared. The resulting sample pad coating liquid contains cocoalkylbis(2-hydroxyethyl)methylammonium chloride at 1.25 wt %.

The sample pad coating liquid was uniformly applied at 0.75 μg/mm² to the glass fiber sample pad. The resulting sample pad was coated with 0.375 μg of cocoalkylbis(2-hydroxyethyl)methylammonium chloride.

Then, the sample pad coated with the sample pad coating liquid was dried with a vacuum dryer to obtain a pretreated sample pad.

Operation was performed in the same manner as Example 66 except as follows: 1.5 wt % Triton X-100 was changed to 2 wt % Triton X-100 in (5) in Example 66; CATINAL SPC-20V-S was not used; and only an influenza A virus positive sample (having an antigen concentration of 10 μg/mL) was prepared and used in (6) in Example 66.

### Example 72

This Example was performed in the same manner as Example 71 except LIPOTHOQUAD C/12 was added at 3.33 wt % to the sample pad coating liquid in (A) in Example 71. Here, the sample pad coating liquid contained cocoalkylbis(2-hydroxyethyl)methylammonium chloride at 2.5 wt %. In addition, the sample pad was coated with 0.75 μg of cocoalkylbis(2-hydroxyethyl)methylammonium chloride.

### Example 73

This Example was performed in the same manner as Example 71 except LIPOTHOQUAD C/12 was added at 6.66 wt % to the sample pad coating liquid in (A) in Example 71. Here, the sample pad coating liquid contained cocoalkylbis(2-hydroxyethyl)methylammonium chloride at 5 wt %. In addition, the sample pad was coated with 1.5 μg of cocoalkylbis(2-hydroxyethyl)methylammonium chloride.

### Comparative Example 42

This Comparative Example was performed in the same manner as Example 71 except the sample pad was not pretreated in (A) in Example 71. Accordingly, the sample pad was not coated with cocoalkylbis(2-hydroxyethyl)methylammonium chloride.

Table 21 shows: the surfactants used to prepare the developing solutions and the sample pads in Examples 71 to 73 and Comparative Example 42; and the evaluation results of the detecting capability.

TABLE 21

| | | | Color Development Intensity [mABS] | | | |
|---|---|---|---|---|---|---|
| | | | Inactivated Influenza A Virus | | | |
| | Developing Solution | Sample Pad | Antigen Concentration: 10 μg/mL | | | |
| | Nonionic Surfactant | Cationic Surfactant | C Line | S Line | B Line | A Line |
| Example 71 | Triton X-100 | cocoalkylbis(2-hydroxyethyl) | 554.2 | N.D. | N.D. | 94.1 |
| | (2 wt %) | methylammonium chloride (0.375 μg) | | | | |
| Example 72 | Triton X-100 | cocoalkylbis(2-hydroxyethyl) | 592.0 | N.D. | N.D. | 127.3 |
| | (2 wt %) | methylammonium chloride (0.75 μg) | | | | |
| Example 73 | Triton X-100 | cocoalkylbis(2-hydroxyethyl) | 727.6 | N.D. | N.D. | 189.9 |
| | (2 wt %) | methylammonium chloride (1.5 μg) | | | | |
| Comparative | Triton X-100 | — | 473.4 | N.D. | N.D. | 41.4 |
| Example 42 | (2 wt %) | | | | | |

As shown in Table 21, the developing solutions in Examples 71 to 73, compared with Comparative Example 42, exhibited a high color development intensity. Any of the test lines corresponding to the respective positive samples were colored, and no test line other than them was colored. This is considered to be because, in Examples, the developing solution contained a nonionic surfactant, the sample pad contained at least an alkylene oxide-addition cationic surfactant, and thus, in the whole, the nonionic surfactant and the alkylene oxide-addition cationic surfactant were present.

### Example 74

This Example was performed in the same manner as Example 1 except as follows: only a positive sample having an antigen concentration of 0.5 ng/ml was prepared as the positive sample in (6) in Example 1, using an immunochromatographic device subjected to 60° C. for eight weeks (that corresponds to room temperature for approximately four years); and a developing solution having no antigen added thereto was used as a negative sample.

### Example 75

This Example was performed in the same manner as Example 1 except as follows: CHAPS (3-[(3-cholamido-propyl)dimethylammonio]-1-propanesulfonate) was further added at 0.1 wt % in (5) in Example 1; only a positive sample having an antigen concentration of 0.5 ng/mL was prepared as the positive sample in (6) in Example 1, using an immunochromatographic device subjected to 60° C. for eight weeks (that corresponds to room temperature for approximately four years); and a developing solution having no antigen added thereto was used as a negative sample.

### Example 76

This Example was performed in the same manner as Example 1 except as follows: 1.5 wt % Triton X-100 in (5) in Example 1 was changed to 1.5 wt % Tween 20; CHAPS (3-[(3-cholamido-propyl)dimethylammonio]-1-propanesulfonate) was further added at 0.1 wt %; only a positive sample having an antigen concentration of 0.5 ng/mL was prepared as the positive sample in (6) in Example 1, using an immunochromatographic device subjected to 60° C. for eight weeks (that corresponds to room temperature for approximately four years); and a developing solution having no antigen added thereto was used as a negative sample.

### Example 77

This Example was performed in the same manner as Example 1 except as follows: sulfobetaine-14 (3-(myristyldimethylammonio)propanesulfonate) was further added at 0.1 wt % in (5) in Example 1; only a positive sample having an antigen concentration of 0.5 ng/ml was prepared as the positive sample in (6) in Example 1, using an immunochromatographic device subjected to 60° C. for eight weeks (that corresponds to room temperature for approximately four years); and a developing solution having no antigen added thereto was used as a negative sample.

### Example 78

This Example was performed in the same manner as Example 1 except as follows: lecithin (derived from soya bean) was further added at 0.1 wt % in (5) in Example 1; only a positive sample having an antigen concentration of 0.5 ng/mL was prepared as the positive sample in (6) in Example 1, using an immunochromatographic device subjected to 60° C. for eight weeks (that corresponds to room temperature for approximately four years); and a developing solution having no antigen added thereto was used as a negative sample.

### Example 79

This Example was performed in the same manner as Example 1 except as follows: AMPHITOL 20AB was further added at 0.33 wt % in (5) in Example 1; only a positive sample having an antigen concentration of 0.5 ng/mL was prepared as the positive sample in (6) in Example 1, using an immunochromatographic device subjected to 60° C. for eight weeks (that corresponds to room temperature for approximately four years); and a developing solution having no antigen added thereto was used as a negative sample. The developing solution prepared in Example 79 contained amidopropylbetaine laurate at 0.1 wt %.

### Example 80

This Example was performed in the same manner as Example 1 except as follows: DMSO (dimethyl sulfoxide) was further added at 0.1 wt % in (5) in Example 1; only a positive sample having an antigen concentration of 0.5 ng/mL was prepared as the positive sample in (6) in Example 1, using an immunochromatographic device subjected to 60° C. for eight weeks (that corresponds to room temperature for approximately four years); and a developing solution having no antigen added thereto was used as a negative sample.

### Example 81

This Example was performed in the same manner as Example 1 except as follows: DMF (N,N-dimethylformamide) was further added at 0.1 wt % in (5) in Example 1; only a positive sample having an antigen concentration of 0.5 ng/mL was prepared as the positive sample in (6) in Example 1, using an immunochromatographic device subjected to 60° C. for eight weeks (that corresponds to room temperature for approximately four years); and a developing solution having no antigen added thereto was used as a negative sample.

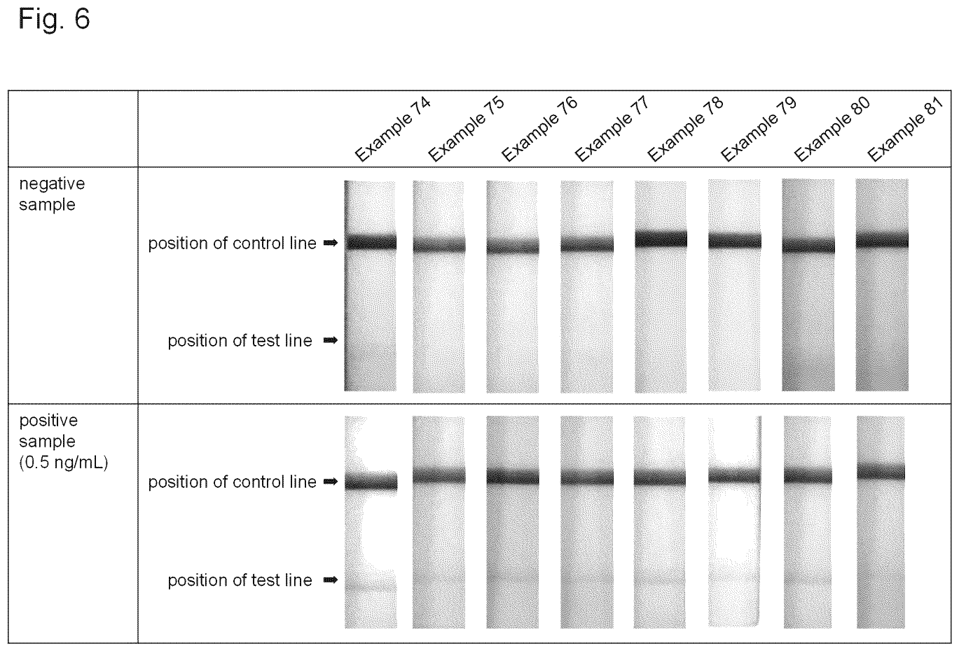

FIG. 6 is the photographs each illustrating the external appearance of the immunochromatographic device in which the negative sample or positive sample produced in each of Examples 74 to 81 was developed. In a case where the positive sample was used in any of Examples 74 to 81 that were evaluated using an immunochromatographic device subjected to accelerated aging at 60° C. to simulate a state created after an elapse of a long period of time, the sample exhibited a color development intensity of 15 mABS or more, so that the coloration of the test line was visually recognized successfully. In a case where the negative sample was used in Example 74, the residual coloration of the gold colloid was left in the background (portion other than the test line and the control line in the membrane), so that some light coloration having a possibility of looking like a test line (5 mABS or more and less than 15 mABS) was observed. However, such coloration was not observed in Examples 75 to 81 performed using a dispersibility improver, thus allowing the risk of erroneous determination about false positiveness to be decreased.

### Example 82

This Example was performed in the same manner as Example 66 except as follows: HAMA serum (HAMA Serum Type II (dissolved in 1 mL of sterile distilled water), manufactured by Roche Diagnostics K.K.) as an interference factor was added for 20-fold dilution in (6) in Example 66; and a developing solution having no antigen added thereto was used as a negative sample.

### Example 83

This Example was performed in the same manner as Example 66 except as follows: CHAPS (3-[(3-cholamido-propyl)dimethylammonio]-1-propanesulfonate) was further added at 0.1 wt % in (5) in Example 66; HAMA serum (HAMA Serum Type II (dissolved in 1 mL of sterile distilled water), manufactured by Roche Diagnostics K.K.) as an interference factor was added for 20-fold dilution in (6) in Example 66; and a developing solution having no antigen added thereto was used as a negative sample.

### Example 84

This Example was performed in the same manner as Example 66 except as follows: mouse IgM was added at 0.1 mg/mL in (5) in Example 66; HAMA serum (HAMA Serum Type II (dissolved in 1 mL of sterile distilled water), manufactured by Roche Diagnostics K.K.) as an interference factor was added for 20-fold dilution in (6) in Example 66; and a developing solution having no antigen added thereto was used as a negative sample.

### Example 85

This Example was performed in the same manner as Example 66 except as follows: mouse IgM was added at 0.1 mg/mL in (5) in Example 66; CHAPS (3-[(3-cholamido-propyl)dimethylammonio]-1-propanesulfonate) was further added at 0.1 wt %; HAMA serum (HAMA Serum Type II (dissolved in 1 mL of sterile distilled water), manufactured by Roche Diagnostics K.K.) as an interference factor was added for 20-fold dilution in (6) in Example 66; and a developing solution having no antigen added thereto was used as a negative sample.

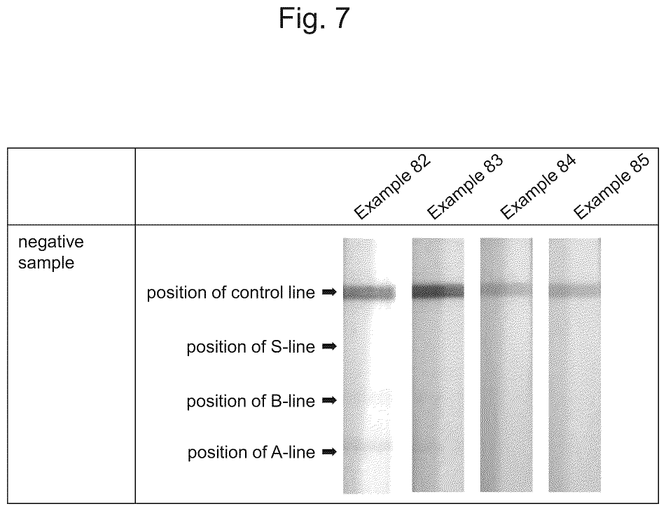

FIG. 7 is the photographs each illustrating the external appearance of the immunochromatographic device in which the negative sample produced in each of Examples 82 to 85 was developed. Example 82 exhibited a color development intensity of 15 mABS or more in the A line, 5 mABS or more and less than 15 mABS in the B line, and less than 5 mABS in the S line, thus verifying false positiveness due to HAMA interference. Example 83 exhibited a color development intensity of 5 mABS or more and less than 15 mABS in the A line and less than 5 mABS in the B line and the S line, thus verifying alleviation of false positiveness due to HAMA interference. In Example 84, no color development intensity was detected in the A line, the B line, or the S line, thus suggesting inhibition of HAMA interference due to the mouse IgM, but the residual coloration of the gold colloid in the background (portion other than the test line and the control line in the membrane) was left in a wide range. In Example 85, no color development intensity was detected in the A line, the B line, or the S line, and no residual coloration of the gold colloid was left in the background. In Examples 83 and 85 performed using a dispersibility improver, no background coloration was observed, and the risk of erroneous determination about false positiveness was successfully decreased.

The upper limits and lower limits of the ranges of values herein described can be arbitrarily combined to define a preferable range. For example, any upper limit and any lower limit of the ranges of values can be combined to define a preferable range. Any upper limits of the ranges of values can be combined to define a preferable range. Any lower limits of the ranges of values can be combined to define a preferable range. In addition, a range of values herein described with “to” includes the values before and after “to” as the lower limit and the upper limit respectively.

It should be understood that expressions in the singular herein include the plural, unless otherwise specified. Accordingly, the singular articles (for example, “a”, “an”, and “the” in English) include plural references unless otherwise specified.

Above, the present embodiments are described in detail, but a specific constitution is not limited to these embodiments, and the present disclosure encompasses any design modification within the scope which does not depart from the spirit of the present disclosure. Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

All publications, patents, and patent applications cited herein are incorporated herein by reference in their entirety.

### REFERENCE SIGNS LIST

1: sample-receiving section
2: labeling substance-retaining section
3: solid-phase support
3a: trapping substance-retaining section
3b: control line
4: absorption pad
5: backing sheet

What is claimed is:

1. A chromatographic developing solution comprising an alkylene oxide-addition cationic surfactant and a nonionic surfactant.

2. The chromatographic developing solution according to claim 1, wherein the alkylene oxide-addition cationic surfactant comprises an ethylene oxide-addition cationic surfactant.

3. The chromatographic developing solution according to claim 1, wherein the alkylene oxide-addition cationic surfactant comprises at least one surfactant selected from a surfactant represented by the following formula (I) and a surfactant represented by the following formula (II): 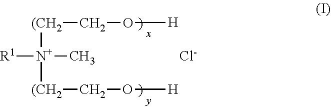
wherein, in the formula (I), R¹ is a C₁₋₃₀ saturated or unsaturated hydrocarbon group, x and y are each independently an integer of 1 to 49, and x+y is 2 to 50, 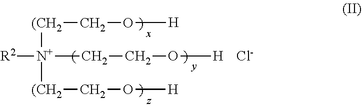
wherein, in the general formula (II), R² is a C₁₋₃₀ saturated or unsaturated hydrocarbon group, x, y, and z are each independently an integer of 1 to 48, and x+y+z is 3 to 50.

4. The chromatographic developing solution according to claim 1, wherein the alkylene oxide-addition cationic surfactant comprises at least one surfactant selected from PEG-5 stearyl ammonium chloride, PEG-2 oleammonium chloride, PEG-2 cocomonium chloride, PEG-15 cocomonium chloride, and PEG-15 steamonium chloride.

5. The chromatographic developing solution according to claim 1, wherein the nonionic surfactant comprises at least one surfactant selected from a surfactant represented by the following formula (III), a surfactant represented by the following formula (IV), a surfactant represented by the following formula (V), a surfactant represented by the following formula (VI), a surfactant represented by the following formula (VII), and a surfactant represented by the following formula (VIII): 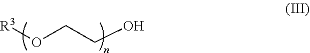
wherein, in the general formula (III), R³ is a C₁₋₃₀ saturated or unsaturated hydrocarbon group, and n is an integer of 1 to 50, 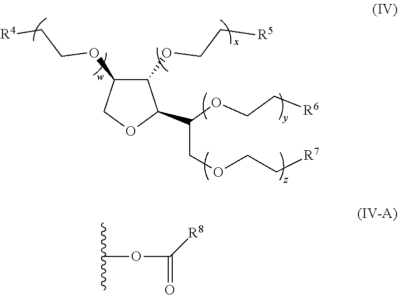
wherein, in the general formula (IV), R⁴ to R⁷ are each independently a group represented by the above-described general formula (IV-A) or a hydroxy group; at least one of R⁴ to R⁷ is a group represented by the above-described general formula (IV-A); at least one of R⁴ to R⁷ is a hydroxy group;
w, x, y, and z are each independently an integer of 1 to 47, and w+x+y+z is 4 to 50; and
in the general formula (IV-A), R⁸ is a C₁₋₃₀ saturated or unsaturated hydrocarbon group; and the wavy line represents a binding site to a carbon atom, 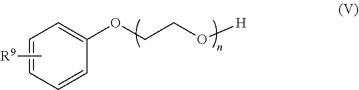
wherein, in the general formula (V), R⁹ is a C₁₋₃₀ saturated or unsaturated hydrocarbon group, and n is an integer of 1 to 50, 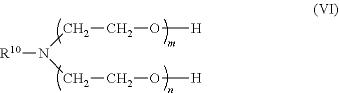
wherein, in the general formula (VI), R¹⁰ is a C₁₋₃₀ saturated or unsaturated hydrocarbon group, m and n are each independently an integer of 1 to 49, and m+n is 2 to 50. 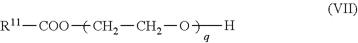
wherein, in the general formula (VII), R¹¹ is a C₁₋₃₀ saturated or unsaturated hydrocarbon group, and q is an integer of 1 to 300, and 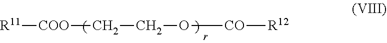
wherein, in the general formula (VIII), R¹¹ and R¹² are each independently a C₁₋₃₀ saturated or unsaturated hydrocarbon group, and r is an integer of 1 to 300.

6. The chromatographic developing solution according to claim 1, wherein a weight ratio of the nonionic surfactant to the alkylene oxide-addition cationic surfactant is 0.14 or more and 40 or less.

7. The chromatographic developing solution according to claim 1, wherein a content of the alkylene oxide-addition cationic surfactant is 0.05 wt % or more and 14 wt % or less.

8. The chromatographic developing solution according to claim 1, wherein a content of the nonionic surfactant is 0.6 wt % or more and 12 wt % or less.

9. The chromatographic developing solution according to claim 1, wherein a HLB value of the alkylene oxide-addition cationic surfactant is 22 or more and 31 or less.

10. The chromatographic developing solution according to claim 1, wherein the chromatographic developing solution is used as a specimen diluent.

11. A kit for detecting an analyte, comprising: the chromatographic developing solution according to claim 1; a chromatograph device including a sample-receiving section; a labeling substance-retaining section; and a solid-phase support, wherein
the labeling substance-retaining section comprises a labeling substance bondable to an analyte, and
the solid-phase support comprises a trapping substance-retaining section, wherein the trapping substance-retaining section comprises a trapping substance bondable to the analyte.

12. A chromatograph device comprising a sample-receiving section, a labeling substance-retaining section, and a solid-phase support, wherein
the labeling substance-retaining section comprises a labeling substance bondable to an analyte,
the solid-phase support comprises a trapping substance-retaining section, wherein the trapping substance-retaining section comprises a trapping substance bondable to the analyte, and
an alkylene oxide-addition cationic surfactant and a nonionic surfactant are each independently in at least one selected from the sample-receiving section and the labeling substance-retaining section.

13. A method for detecting an analyte contained in a sample, comprising the following (1) and (2):
(1) developing a mobile phase in the presence of an alkylene oxide-addition cationic surfactant and a nonionic surfactant in a solid-phase support of a chromatograph device, wherein the mobile phase comprises the sample and a labeling substance bondable to an analyte; and
(2) detecting the analyte in the developed mobile phase in a trapping substance-retaining section, wherein the trapping substance-retaining section comprises a trapping substance bondable to the analyte in the solid-phase support.

14. A kit comprising:
the chromatograph device of claim 12 and a chromatographic developing solution, wherein the alkylene oxide-addition cationic surfactant and the nonionic surfactant are each independently in at least one selected from the chromatographic developing solution, the sample-receiving section, and the labeling substance-retaining section.

15. The kit according to claim 14, wherein the labeling substance comprises colored particles.
